(19) United States
(12) Patent Application Publication
(10) Pub. No.: US 2021/0220648 A1
(43) Pub. Date: Jul. 22, 2021

(54) NERVE STIMULATION APPARATUS AND NERVE STIMULATION METHOD

(71) Applicant: ADRIAKAIM INC., Tokyo (JP)

(72) Inventor: Hiroyuki IMABAYASHI, Tokyo (JP)

(21) Appl. No.: 16/810,003

(22) Filed: Mar. 5, 2020

(30) Foreign Application Priority Data
Jan. 21, 2020 (JP) 2020-007854

(51) Int. Cl. A61N 1/36; A61N 1/375; A61B 5/024; A61B 5/00

(52) U.S. Cl. CPC A61N 1/36053; A61N 1/37516; A61N 1/36175; A61B 5/742; A61B 5/024

(57) ABSTRACT

A nerve stimulation apparatus includes: an intravascular stimulation electrode that transfers electric energy from inside of a blood vessel to a vagus nerve outside of the blood vessel; an intravascular heartbeat detection electrode that detects heartbeat information inside the blood vessel; and a notification member, in which calculating a heart rate reduction rate on the basis of a pre-stimulation heart rate HR_base based on the heartbeat information detected prior to output of the electric energy and an in-stimulation heart rate HR_st based on the heartbeat information detected during output of the electric energy; making the notification member notify the heartbeat status in a case in which at least the heart rate reduction rate is less than the first threshold value; and stops the stimulus output generating circuit from outputting the electric energy in a case in which the heart rate reduction rate is no as than the first threshold value.

This application is based on and claims the benefit of priority from Japanese Patent Application No. 2020-007854, filed on 21 Jan. 2020, the content of which is incorporated herein by reference.

### BACKGROUND OF THE INVENTION

### Field of the Invention

The present invention relates to a nerve stimulation apparatus and a nerve stimulation method.

### Related Art

Conventionally, a medical instrument for stimulating a nerve from inside of a blood vessel has been known. For example, Japanese Unexamined Patent Application (Translation of PCI Application), Publication No. 2010-516405 discloses a medical electrical lead for nerve stimulation and a configuration delivering an electrical pulse across a vessel wall.

Patent Document 1: Japanese Unexamined Patent Application (Translation of PCT Application), Publication No. 2010-516405

### SUMMARY OF THE INVENTION

Japanese Unexamined Patent Application (Translation of PCT Application), Publication No. 2010-516405 discloses that a lead body can be further rotated or positioned until a maximum or optimum electrical stimulation threshold by electrodes has been achieved across the vessel wall to the adjacent nerve or muscle to be stimulated, and that a stimulating pulse delivered by the electrodes cap then be measured to determine if an optimal stimulation threshold has been reached. However, a further improvement is desired in order to practice nerve stimulation with greater safety.

The present invention has been made in view of the aforementioned problem, and an object of the present invention is to provide a nerve stimulation apparatus of greater safety.

A nerve stimulation apparatus according to the present invention includes:

an intravascular stimulation electrode that transfers electric energy from inside of a blood vessel to a vagus nerve outside of the blood vessel; an intravascular heartbeat detection electrode that detects heartbeat information inside the blood vessel; and a stimulus output generating circuit that is connected to the intravascular stimulation electrode through a conductor and outputs the electric energy to the intravascular stimulation electrode; a heart rate detection circuit that is connected to the intravascular heartbeat detection electrode through a conductor and detects a heart rate on the basis of the heartbeat information detected by the intravascular heartbeat detection electrode; a heartbeat status determination circuit that determines a heartbeat status on the basis of the heart rate detected by the heart rate detection circuit; and a notification member that notifies the heartbeat status determined by the heartbeat status determination circuit, in which the heartbeat status determination circuit: calculates a heart rate reduction rate on the basis of a pre-stimulation heart rate HR_base detected by the heart rate detection circuit before the stimulus output generating circuit starts outputting the electric energy, and an in-stimulation heart rate HR_st detected by the heart rate detection circuit while the stimulus output generating circuit outputs the electric energy; makes the notification member notify the heartbeat status in a case in which at least the heart rate reduction rate is less than a first threshold value; and stops the stimulus output generating circuit from outputting the electric energy in a case in which the heart rate reduction rate is no less than the first threshold value.

According to the present invention, a nerve stimulation apparatus of greater safety can be provided.

### BRIEF DESCRIPTION OF THE DRAWINGS

FIG. 1 is a schematic view showing an entire configuration of a medical instrument set including the nerve stimulation apparatus according to an embodiment of the present invention;

FIG. 2 is a schematic view showing a configuration of an electrical stimulation electrode of the medical instrument set including the nerve stimulation apparatus according to the embodiment of the present invention;

FIG. 3 is a schematic perspective view showing an electrode support fixation member of a lead portion and a distal end portion or an operation sheath of the medical instrument set including the nerve stimulation apparatus according to the embodiment of the present invention;

FIG. 4 is a schematic front view showing a configuration of a biasing member of the electrical stimulation electrode in the medical instrument set including the nerve stimulation apparatus according to the embodiment of the present invention;

FIG. 5 is a lateral view from the direction of the A arrow in FIG. 4;

FIG. 6 is a schematic cross-sectional view taken along a longitudinal direction of the biasing member of the medical instrument set including the nerve stimulation apparatus according to the embodiment of the present invention;

FIG. 7 is a cross-sectional view taken along the line B-B in FIG. 6;

FIG. 8 is a schematic cross-sectional view showing a configuration of a base end side of the operation sheath of the medical instrument set including the nerve stimulation apparatus according to the embodiment of the present invention;

FIG. 9 is a schematic cross-sectional view showing a state in which the biasing member is inserted into a removal sheath of the medical instrument set including the nerve stimulation apparatus according to the embodiment of the present invention;

FIG. 10 is a schematic cross-sectional view showing a configuration of the removal sheath of the medical instrument set including the nerve stimulation apparatus according to an embodiment of the present invention;

FIG. 11A is a block diagram schematically showing an electrical circuit and the like provided in an electrical stimulation device of the nerve stimulation apparatus according to the embodiment of the present invention;

FIG. 11B is a schematic view showing an example of a stimulating pulse generated by the electrical stimulation device of the nerve stimulation apparatus according to the embodiment of the present invention;

FIG. 11C is a schematic view showing, as an example of the stimulating pulse, a pulse shape of the continuous stimulation mode;

FIG. 11D is a schematic view showing, as an example of the stimulating pulse, a pulse shape of the intermittent stimulation mode;

FIG. 11E is a schematic view showing heart rate variation in the intermittent stimulation mode;

FIG. 12 is a schematic view showing a state in which the biasing member of the medical instrument set including the nerve stimulation apparatus according to the embodiment of the present invention is placed in the superior vena cava;

FIG. 13A is a schematic view showing a placement operation of the electrical stimulation electrode of the medical instrument set including the nerve stimulation apparatus according to the embodiment of the present invention;

FIG. 13B is a schematic view showing a placement operation of the electrical stimulation electrode of the medical instrument set including the nerve stimulation apparatus according to the embodiment of the present invention;

FIG. 14A is a schematic view showing a placement operation of the electrical stimulation electrode of the medical instrument set including the nerve stimulation apparatus according to the embodiment of the present invention;

FIG. 14B is a schematic view showing a placement operation of the electrical stimulation electrode of the medical instrument set including the nerve stimulation apparatus according to the embodiment of the present invention;

FIG. 15 is a schematic view showing a placement operation of the electrical stimulation electrode of the medical instrument set including the nerve stimulation apparatus according to the embodiment of the present invention.

FIG. 16 is a schematic view showing an example of slack of a linear body of the medical instrument set including the nerve stimulation apparatus according to an embodiment of the present invention; and

FIG. 17 is a schematic view showing an example of slack of the linear body of the medical instrument set including the nerve stimulation apparatus according to an embodiment of the present invention.

### DETAILED DESCRIPTION OF THE INVENTION

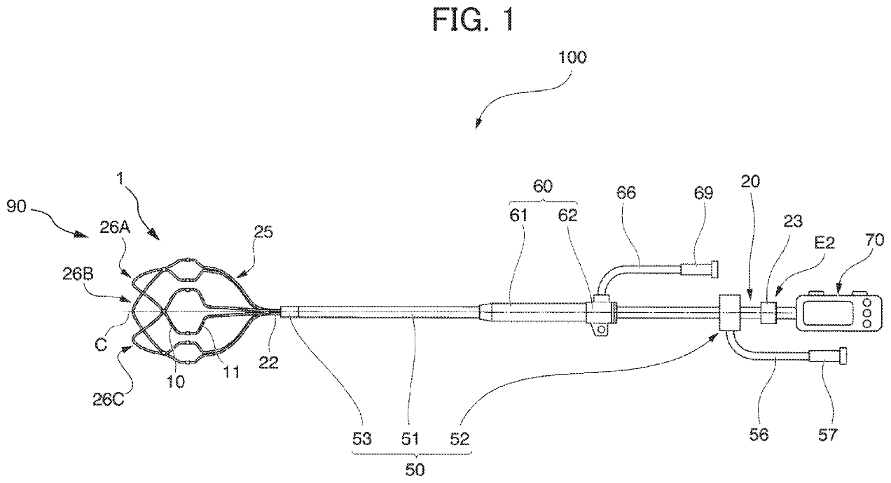

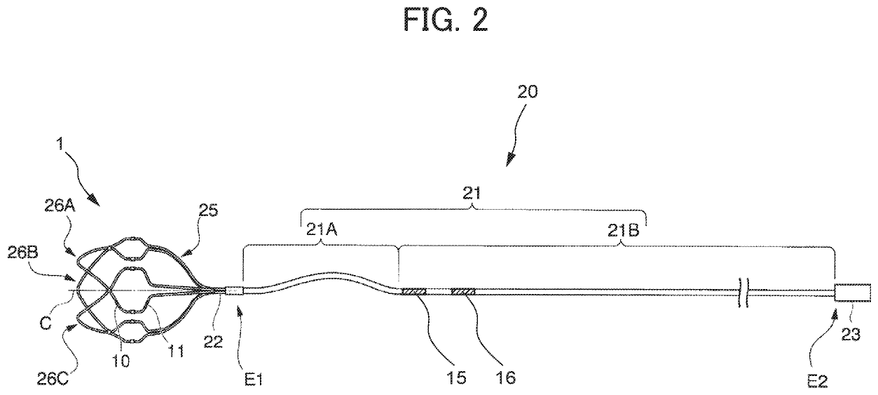

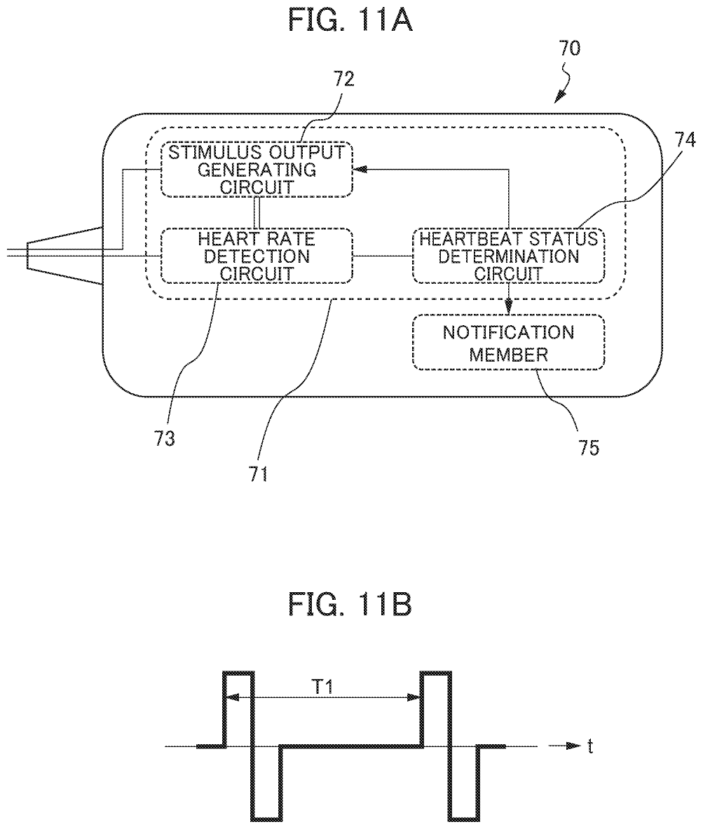

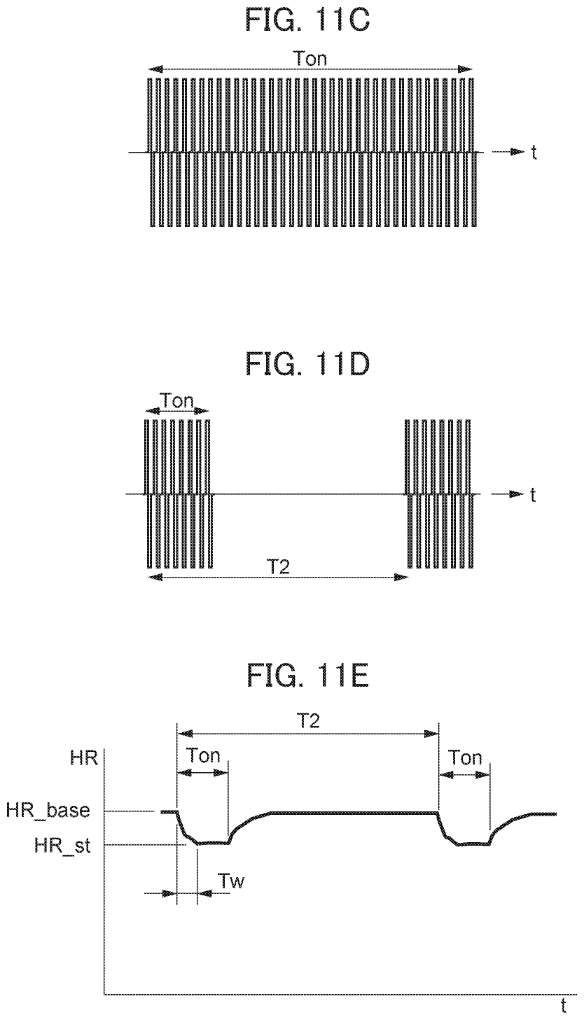

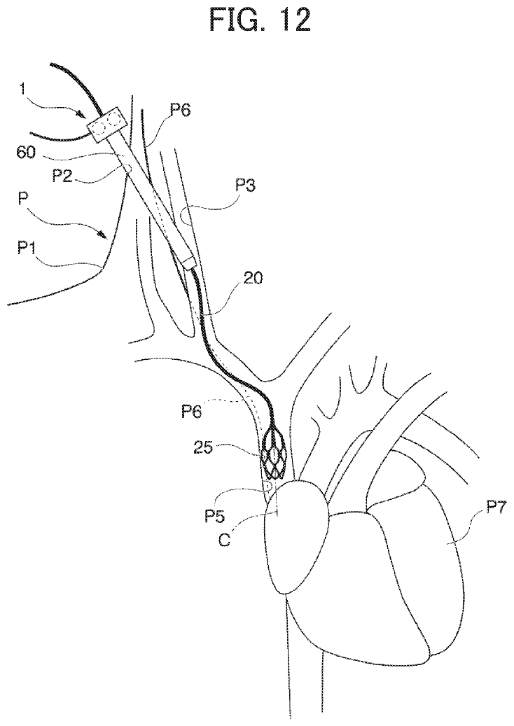

Hereinafter, an embodiment of the present invention is described with reference to the drawings. The drawings are schematic views and shapes and dimensions are exaggerated. The nerve stimulation apparatus 90 of the present embodiment includes: stimulation electrode portions 10, 11 as the intravascular stimulation electrode; and heartbeat detection electrodes 15, 16 as the intravascular heartbeat detection electrode (see FIG. 2). The nerve stimulation apparatus 90 further includes a stimulus output generating circuit 72, a heart rate detection circuit 73, a heartbeat status determination circuit 74, and a notification member 75 (see FIG. 11A). In the present embodiment, the stimulation electrode portions 10, 11 and the heartbeat detection electrodes 15, 16 are provided in an electrical stimulation electrode 1 (medical instrument) shown in FIG. 1. The stimulus output generating circuit 72, the heart rate detection circuit 73, the heartbeat status determination circuit 74, and the notification member 75 are provided in an electrical stimulation device 70 shown in FIG. 1. As shown in FIG. 1, the nerve stimulation apparatus 90 is used together with an operation sheath 50 and a removal sheath 60. The nerve stimulation apparatus 90, the operation sheath 50 and the removal sheath 60 constitute a medical instrument set 100.

The electrical stimulation electrode 1 is used in a state of being inserted into a blood Vessel in order to stimulate a nerve from an inner wall of the blood vessel. The electrical stimulation electrode 1 is used together with the electrical stimulation device 70 that applies to the electrical stimulation electrode 1 a stimulating pulse as the electric energy. As shown in FIG. 2, the electrical stimulation electrode 1 is provided with: the stimulation electrode portions 10, 11 as the intravascular stimulation electrode that transfers electric energy from inside of the blood vessel to a vagus nerve outside of the blood vessel; a lead portion 20 (linear body); an electrode support 25 (biasing member); and the heartbeat detection electrodes 15, 16 as the intravascular heartbeat detection electrode that detects heartbeat information inside the blood vessel.

The stimulation electrode portions 10, 11 applies an electrical stimulus through the inner wall of the blood vessel. One of the stimulation electrode portions 10, 11 serves as a positive electrode, while the other serves as a negative electrode. The lead portion 20 is linearly formed, and a wiring 35 (described later, see FIG. 6) electrically connected to the stimulation electrode portions 10, 11 is inserted through the lead portion 20. The electrode support 25 (described later) is provided at a first end portion E1 of the lead portion 20 (tip end of the linear body). As shown in FIG. 1, the electrical stimulation device 70 is connected to a second end portion E2 of the lead portion 20.

The heartbeat detection electrodes 15, 16 detect an electrocardiographic waveform through blood. One of the heartbeat detection electrodes 15, 16 serves as a positive electrode, while the other serves as a negative electrode. The heartbeat detection electrodes 15, 16 are provided to be exposed on the lead portion 20 of the electrical stimulation electrode 1, and a wiring (not illustrated) is electrically connected to the heartbeat detection electrodes 15, 16. The wiring is inserted into the lead portion 20 and connected to the second end portion E2 of the lead portion 20.

The electrode support 25 is arranged at the tip end of the lead portion 20, and elastically presses the inner wall of the blood vessel when the electrical stimulation electrode 1 is inserted into the blood vessel. In the present embodiment, the electrode support 25 is formed in a basket-like shape from linear elastic members 26A, 26B, 26C, so as to be substantially rotationally symmetric about a predetermined axis line C. As used herein, the “basket-like shape” means a solid shape formed from the linear members, with an opening on the tip end side, of which external shape gradually reduces in diameter toward the base end side. The tip end side may be in a substantially cylindrical shape. The base end side may be in a conical shape or a quasi-conical shape of which external shape gradually reduces in diameter toward the base end side. The “quasi-conical shape” means a shape of which lateral face is bulging or constricted compared to a perfect conical shape.

The elastic member 26A is provided with the stimulation. electrode portions 10, 11. The electrode support 25 is fixed at the first end portion E1 of the lead portion 20 through an electrode support fixation member 22 (described later). Hereinafter, for describing a relative on of each member of the electrical stimulation electrode 1 along the longitudinal direction of the electrical stimulation electrode 1, a position close to the electrode support 25 may be referred to as the “tip end side”, while a position close to the electrical stimulation device 70 may be referred to as the “base end side”. In regard to the electrode support 25, a distal side with respect to the lead portion 20 may be referred to as the “tip end side”, while a proximal side with respect to the lead portion 20 may be referred to as the “base end side”.

The lead portion 20 includes a lead main body 21, the electrode support fixation member 22, and a connector 23. The lead main body 21 is a linearly extending tubular member formed from a biologically compatible material such as a polyamide resin. For example, an external diameter of the lead main body 21 is no less than 0.8 mm and no greater than 2 mm. For example, a length of the lead main body 21 is no less than 500 mm and no greater than 1000 mm. A wiring 35 (described later, not illustrated) is inserted into a pipeline of the lead main body 21.

In addition to the wiring 35 as a conductor connected to the stimulation electrode portions 10, 11 and the wiring as a conductor connected to the heartbeat detection electrodes 15, 16, a reinforcement member (tension member) such as a metal wire may be arranged in, or inserted into, the lead main body 21, in order to increase tensile strength of the lead main body 21. In the case in which the reinforcement member is arranged or inserted, even when a tensile force is applied to the lead main body 21, breaking and the like of the wiring 35 may be prevented since the reinforcement member can bear a load of the tensile force. The surface of the lead main body 21 may be provided with an appropriate coating. For example, the surface of the lead main body 21 may be provided with an antithrombotic coating. For example, the surface of the lead main body 21 may be provided with a coating that improves slidability. It is to be noted that the heartbeat detection electrodes 15, 16 are not to be provided with the antithrombotic coating which is a non-conductor.

As shown in FIG. 2, the lead main body 21 is provided with a soft portion 21A and a non-soft portion 21B, in this order from the tip end side toward the base end side. The soft portion 21A is formed within a predetermined scope from the tip end side of the lead main body 21. The soft portion 21A has a first bendabjlity. The non-soft portion 21B is formed within a predetermined scope on the base end side of the lead main body 21, outside of the soft portion 21A. The non-soft portion 21B has a second bendability smaller than the first bendability (less bendable).

If the lead main body 21 is stiff all over, an external force due to movement such as a body motion of a patient after the placement may be transmitted to, and in turn displace, the electrode support 25 placed in the blood vessel. When the electrode support 25 is displaced, the nerve stimulation effect may be reduced or lost. However, since the soft portion. 21A, which is a part of the lead main body 21, is sufficiently soft, the lead main body 21 can be placed in the blood vessel with the soft portion 21A being slack. As a result, the soft portion 21A can absorb the external force generated on the base end side and prevent the electrode support 25 from displacing. The “first bendability” means such bendability that, for example, slack can absorb, inside the blood vessel, the external force generated on the base end side due to a body motion and the like. In order to impart the first bendability to the soft portion 21A, the soft portion 21A may be formed from, for example, a material having Shore hardness of about no less than 20D and no greater than 40D. Furthermore, in order to impart the first bendability to the soft portion 21A, the soft portion 21A may be made to have a sufficient length for absorbing the external force due to body motion and the like.

The length for a obtaining the first bendability depends on the position at which the electrode support 25 is placed. For example, in the case of inserting the electrode support 25 from the vena jugularis interna and placing in the superior a cava, the length of the soft portion 21A may be about no less than 50 mm and no greater than 250 mm. Also in the case of inserting the electrode support 25 from the subclavian vein, the length of the soft portion 21A may be about no less than 50 mm and no greater than 250 mm. The longer soft portion 21A is more desirable in order that the soft portion 21A may absorb the external force. For example, the length of the soft portion 21A no less than 100 mm enables superior absorption of the external force, regardless of whether the electrode support 25 is placed via the vena jugularis interna, or via the subclavian vein.

The “second bendability” of the non-soft portion 21B means such rigidity that a force applied at the base end portion of the non-soft portion 21B in the axis line direction of the non-soft portion 21B can be transmitted to the tip end portion of the non-soft portion 21B.

Preferably, the heartbeat detection electrodes 15, 16 are provided in the non-soft portion 21B of the lead portion 20. This stabilizes the positional relationship of the heartbeat detection electrodes 15, 16, and in turn, enables stable capture of the heartbeat information. In addition, in this configuration, the wiring of the heartbeat detection electrodes 15, 16 is not inserted into the soft portion 21A in addition to the wiring 35 of the stimulation electrode portions 10, 11. The softness of the soft portion 21A can thus be ensured. More preferably, the heartbeat detection electrodes 15, 16 are provided in the vicinity of the tip end portion of the non-soft portion 21B of the lead portion 20. This enables stable capture of the heartbeat information at a position as close to the stimulation electrode portions 10, 11 as possible.

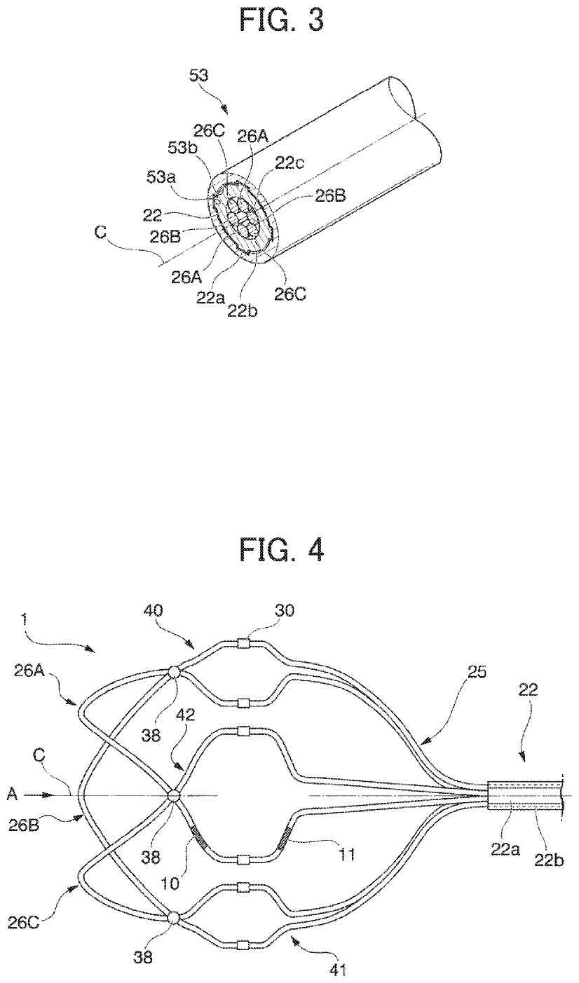

The electrode support fixation member 22 is a tubular member connected to the lead main body 21 at the first end portion E1, which is the tip end side of the lead portion 20. The center axis line of the electrode support fixation member 22 is coaxial with the axis line C. As shown in FIG. 3, a through-hole 22c is formed in a center of the electrode support fixation member 22 to extend in the longitudinal direction. Base end portions of the elastic members 26A, 26B, 26C (described later) are inserted from the tip end side of the through le 22c. The wiring 35 (not illustrated) extending from the elastic member 26A is inserted from the base end side (not illustrated) of the through-hole 22c.

On the outer periphery of the electrode support fixation member 22, an engaging projection 22a protrudes from an outer peripheral surface 22b, which is a cylindrical face. A shape of the engaging projection 22a is not particularly limited as long as it is a non-circular shape that can detachably engage with an engaging portion 53 of the operation sheath 50 (described later) around the axis line C. In the present embodiment, for example, the engaging projection 22a extends in the longitudinal direction of the electrode support fixation member 22, with the outer peripheral surface 22b partially protruding outward in the radial direction. In the present embodiment, the engaging projection 22a is provided at four positions dividing the outer peripheral surface 22b into four in the circumferential direction. The electrode support fixation member 22 may be formed from a metal superior in biological compatibility such as titanium. The surface of the electrode support fixation member 22 may be provided with an antithrombotic coating.

As shown in FIG. 1, the connector 23 connects the electrical stimulation device 70 at the second end portion E2 of the lead portion 20. The connector 23 electrically connects between the wiring 35 extending to the base end side of the lead portion 20 and the electrical stimulation device 70. As the connector 23, for example, the well-known IS-1 connector, other waterproof connectors, and the like may be used. It is to be noted that the connector 23 is not essential for the electrical stimulation electrode 1, and the wiring 35 inserted into the lead portion 20 and the electrical stimulation device 70 may be directly connected.

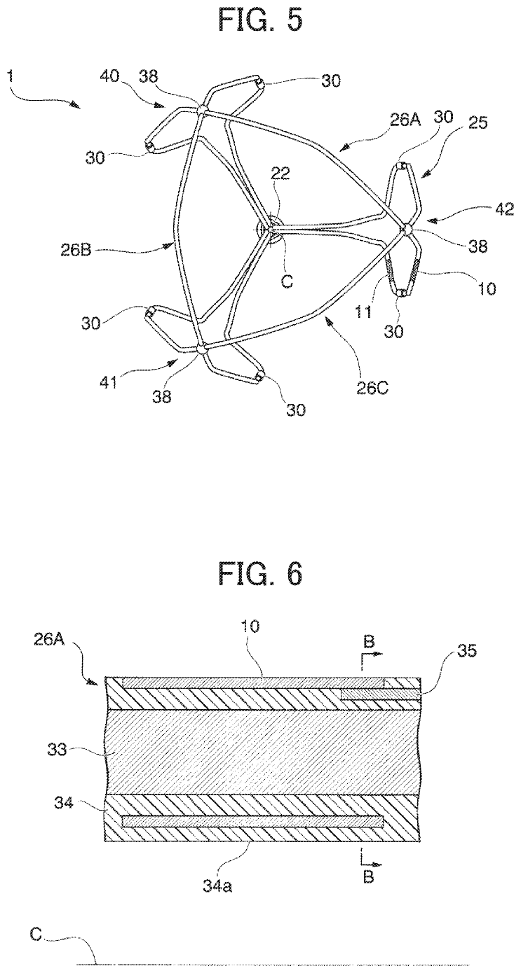

Hereinafter, the external shape and the inner structure of the electrode support 25 are described. As shown in FIGS. 4 and 5, the electrode support 25 is provided with the elastic members 26A, 26B, 26C that are bent in the same shape, for example. The elastic members 26A, 26B, 26C are arranged out of alignment in the circumferential direction about the axis line C. In the present embodiment, the external shape of the electrode support 25 is a substantially cylindrical shape on the tip end side, and a bulging quasi-conical shape on the base end side. In other words, the external shape of the electrode support 25 is a cannonball-like shape having an apex on the base end side.

The elastic members 26A, 26B, 26C are each provided with a projection 30 protruding from the external surface thereof in order to increase a frictional force acting on the inner wall of the blood vessel. The elastic member 26A is provided with the stimulation electrode portions 10, 11. The elastic members 26B, 26C have external shapes similar to that of the elastic member 26A except that the stimulation electrode portions 10, 11 are not provided.

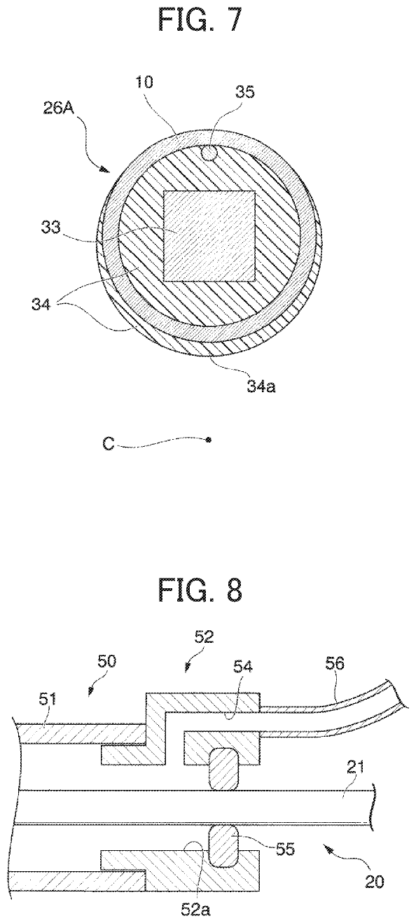

As shown in FIGS. 6 and 7, the elastic member 26A is constituted of a linear member in which the outer periphery of a wire portion 33 is covered with an electrically insulating cover 34. A cross-sectional shape of the wire portion 33 perpendicular to the longitudinal direction is not particularly limited. The cross-sectional shape of the wire portion 33 perpendicular to the longitudinal direction may be, for example, circular, oval, square, rectangular, and the like. An external diameter or a side dimension of the wire portion 33 may be about no less than 0.2 mm and no greater than 0.5 mm. In the present embodiment, the cross section of the wire portion 33 perpendicular to the longitudinal direction is formed in a square of 0.3 mm×0.3 mm, for example. As a material for the wire portion 33, a shape-memory alloy, a hyperelastic wire and the like may be used. A resin coating film may be provided on the outer periphery of the wire portion 33. As a material for the resin coating film, for example, a polyurethane resin, a polyamide resin, a fluorocarbon resin and the like may be used. A thickness of the resin coating film may be, for example, no less than 50 μm and no greater than 500 μm.

The cross-sectional shape of the cover 34 perpendicular to the longitudinal direction is, for example, circular. The cross-sectional shape of the cover 34 is not limited to the circular shape and may also be oval and the like. The external diameter of the cover 34 is, for example, 0.8 mm. As a suitable material for the cover 34, for example, a resin such as a polyurethane resin, a polyamide resin, and the like may be used.

The stimulation electrode portion 10 is formed from a biologically compatible metal such as a platinum-iridium alloy and the like, in a cylindrical shape. The stimulation electrode portion 10 has, for example, an external diameter of 0.8 mm and a length of 4 mm. A part of the outer peripheral surface of the stimulation electrode portion 10 is exposed from the cover 34. An exposed area of the stimulation electrode portion 10 may be, for example, no less than 1 mm² and no greater than 5 mm². The stimulation electrode portion 10 is exposed toward a radially outer side of the electrode support 25 when the elastic member 26A is assembled to the electrode support 25. The “radially outer side of the electrode support 25” is a direction. away from the axis line C along a line perpendicular to the axis line C. The stimulation electrode portion 10 and the wire portion 33 are electrically insulated from each other by the cover 34. In order to ensure the electrical insulation between the cover 34 and the wire portion 33, an insulating member made of a resin may be provided between the stimulation electrode portion 10 and the wire portion 33. The wiring 35 is electrically connected to the inner peripheral surface of the stimulation electrode portion 10 by means of welding and the like. The wiring 35 extends along the wire portion 33 inside the cover 34, and protrudes from the base end portion of the elastic member 26A toward the lead portion 20.

For example, the wiring 35 may be formed by covering a twisted wire made of a flex-resistant nickel-cobalt alloy with an electrically insulating material. The nickel-cobalt alloy is exemplified by 35NLT25%Ag, 35NLT28%Ag, and 35NLT41%Ag. The electrically insulating material for the wiring 35 is exemplified by FIFE (ethylene-tetrafluoroethylene copolymer resin) of 20 μm thickness, PTFE (polytetrafluoroethylene resin) of 20 μm thickness, and the like.

Although not illustrated, the stimulation electrode portion 11 has the same configuration as the stimulation electrode portion 10, except for being formed in a site on the base end side, facing the stimulation electrode portion 10. The stimulation electrode portions 10, 11 are arranged at an interval of about at least 3 mm to 20 mm in a direction along the axis line C. FIG. 6 shows the wiring 35 connected to the stimulation electrode portion 10, while another wiring 35 having similar configuration is connected to the stimulation electrode portion 11. The two wirings 35 are arranged parallel to each other inside the cover 34 (described later), more on the base end side of the elastic member 26A. than the stimulation electrode portion 11. The wirings 35 are electrically insulated from each other.

The heartbeat detection electrodes 15, 16 are each formed from a biologically compatible metal such as a platinum-iridium alloy and the like, in a cylindrical shape. The heartbeat detection electrodes 15, 16 are formed to correspond to the external diameter of the lead portion, and 6 mm in length. The heartbeat detection electrodes 15, 16 are insulated from each other by the lead portion and arranged at an interval of about at least 5 mm to 20 mm in a direction along the axis line C. A wiring (not illustrated) is electrically connected to the inner peripheral surface of the heartbeat detection electrodes 15, 16 by means of welding and the like. The wiring is inserted into the lead portion. For example, the wiring may be formed by covering a twisted wire made of a flex-resistant nickel-cobalt alloy with an electrically insulating material and is identical to the wiring 35 used for the stimulation electrode.

The elastic members 26A, 26B, 26C configured as described above are elastic due to the wire portion 33 and the like. As shown in FIGS. 4 and 5, in the electrode support 25, adjacent two of the elastic members 26A, 26B, 26C intersect with each other. An intersection of the elastic members 26A, 26B, an intersection. of the elastic members 26B, 26C, and an intersection of the elastic members 26C, 26A are fixed by an elastic member fixing portion 38. The elastic member fixing portion 38 can be formed by, for example, joining the cover 34 of each of the elastic members 26A, 26B, 26C through melting. The electrode support 25 is provided with three elastic member fixing portions 38. The elastic members 26A, 26B, 26C more on the tip end side than the elastic member fixing portion 38 form a substantially hexagonal opening viewed from the direction along the axis line C, as shown in FIG. 5. As shown in FIG. 4, protruding portions 40, 41, 42 are provided in a middle part of the electrode support 25 in the direction along the axis line C, more on the base end side than the elastic member fixing portion 38.

As shown in FIG. 5, the protruding portions 40, 41, 42 constitute an outer periphery of the electrode support 25 in a natural state in which the external diameter is the greatest. As used herein, the “natural state” of the electrode support 25 means an assembled state in which no external force acts thereupon, or deformation caused by an external force is ignorable.

The external diameter of the electrode support 25 in the natural state is greater than an internal diameter of a blood vessel such as the superior vena cava in which the electrode support 25 is to be placed. For example, in the case in which the blood vessel is the superior vena cava, the external diameter of the electrode support 25 in the natural state may be no less than 20 mm and no greater than 40 mm. The length of the electrode support 25 in the direction of the axis line C is, for example, 5 mm. The base end portions of the elastic members 26A, 26B, 26C of the electrode support 25 are connected to the electrode support fixation member 22 of the lead portion 20 through welding, adhesion, or swaging.

The electrode support 25 configured as described above reduces in diameter in the radial direction compared to the natural state when, for example, a radially inward external force is applied thereto through the protruding portions 40, 41, 42 and the like. In this case, the axial length of the electrode support 25 is along the axis line C. The elastic members 26A, 26B, 26C arranged adjacently in the circumferential direction approach each other in the circumferential direction.

When the electrode support 25 is to be placed in a blood vessel, the operation sheath 50 shown in FIG. 1 enables the electrode support 25 to move back and forth and rotate in the blood vessel. The operation sheath 50 is provided with the engaging portion 53, a sheath main body 51 (first cylindrical portion), and a hub 52, in this order from the tip end side.

As shown in FIG. 3, the engaging portion 53 engages with the electrode support fixation member 22 of the lead portion 20 in the circumferential direction. The inner periphery of the engaging portion 53 has convexity and concavity corresponding to convexity and concavity on the outer periphery of the electrode support fixation member 22. In the present embodiment, the inner periphery of the engaging portion 53 is provided with an inner peripheral surface 53b and an engaging groove 53a. The inner peripheral surface 53b is a cylindrical face that slidably fits over the outer peripheral surface 22b of the electrode support fixation member 22 in the direction along the axis line C. The engaging groove 53a is formed on the inner peripheral surface 53b and fits the engaging projection 22a of the electrode support fixation member 22. The engaging groove 53a extends in the axis line direction (longitudinal direction) of the operation sheath 50, as the engaging projection 22a extends in the axis line direction (along the axis line C) of the electrode support fixation member 22. As a result, the engaging portion 53 is detachable in the axis line direction of the electrode support fixation member 22. The electrode support fixation member 22 inserted into the engaging portion 53 is engaged with the operation sheath 50 to be movable in the axis line direction and immovable in the circumferential direction. In a state in which the electrode support fixation member 22 engages with the engaging portion 53, rotating the operation sheath 50 about the axis line thereof transmits rotary torque to the electrode support 25.

The engaging portion 53 may be formed integrally with the sheath main body 51 (described later), or by deforming the sheath main body 51. It is to be noted that the engaging portion 53 may be formed as a separate member from the sheath main body 51, as long as it is fixed to the sheath main body 51.

As shown in FIG. 1, the sheath main body 51 is a cylindrical member through which the lead portion 20 is inserted. The sheath main body 51 is slidable with respect to the lead portion 20. The engaging portion 53 is arranged at the tip end portion of the sheath main body 51, coaxially with the sheath main body 51. The hub 52 (described later) is fixed to the base end portion of the sheath main body 51. The sheath main body 51 is formed from a biologically compatible material such as a polyurethane resin, a polyamide resin, a polyethylene resin, a fluorocarbon resin, and the like. The sheath main body 51 has such torsional rigidity that rotary torque generated by rotating the base end portion by hand, for example, can be transmitted to the engaging portion 53.

For example, a metal blade of stainless steel, tungsten, and the like may be enclosed in the resin of the sheath main body 51. Rigidity and kink resistance or the sheath main body 51 can thus be improved. An appropriate coating film may be provided on the surface of the sheath main body 51 as needed The coating film is exemplified by a coating film improving an antithrombotic property and a coating film improving slidability. An external diameter of the sheath main bod 51 may be, for example, no less than 2.0 mm and no greater than 2.9 mm. An internal diameter of the sheath main body 51 may be, for example, no less than 1.0 mm and no greater than 2.5 mm. The sheath. main body 51 has such a length that the engaging portion 53 can be arranged in the vicinity of a position at which the electrode support 25 is placed. The length of the sheath main body 51 may be no less than 300 mm and no greater than 400 mm. In the case in which the sheath main body 51 has such a shape and a length, for obtaining such torsional rigidity that the rotation operation can be performed with respect to the electrode support fixation member 22, the Shore hardness of a material used for the sheath main body 51 may be about no less than 55D and no greater than 70D. In the case of enclosing the metal blade in the sheath main body 51, a material with lower Shore hardness can be used.

As shown FIG. 8, the hub 52 is a substantially cylindrical member. The hub 52 is provided with a through-hole 52a that extends in the axis line direction and communicates with an inner space of the sheath main body 51, and a side hole 54 branched from the through hole 52a. A sealing member 55 such as an O-ring is provided to the through-hole 52a. The sealing member 55 provides watertight sealing of a periphery of the lead main body 21 of the lead portion 20 inserted through the operation sheath 50, whereby leakage of blood and the like is prevented. A tube 56 is connected to the side hole 54. A connector 57 such as a Luer-Lok connector is provided at the other end of the tube 56 (see FIG. 1). By connecting the connector 57 to a source of a medicinal solution such as physiological saline with heparin, the medicinal solution such as physiological saline with heparin can be supplied to the operation sheath 50 through the tube 56 and the side hole 54. It is to be noted that the tube 56 is not essential, and a connector or the like, to which an administration tube etc. can be connected, may provided directly to the side hole 54.

The hub 52 arranged outside the patient's body. Therefore, resin such as a silicone resin, a polycarbonate resin, and an ABS (acrylonitrile butadiene styrene) resin may be used as a material for the hub 52.

The operation sheath 50 is removed from the blood vessel after completion of placement of the electrode support 25 described later. In this case, the operation sheath 50 may be removed by using a surgical instrument such as scissors, or the operation sheath 50 may be configured as a peel-away sheath that can be removed by hand. The peel-away configuration is exemplified by a configuration in which the orientation of the resin corresponds to the axis line direction of the operation sheath 50, and the resin can thus be torn along the axis line direction. The peel-away configuration is also exemplified by a configuration in which a low-strength portion such as a groove serving as a tear line is provided on the outer periphery of the operation sheath 50 along the axis line direction of the operation sheath 50.

The removal sheath 60 shown in FIG. 1 is a member that holds the electrode support 25 in a contracted state when the electrode support 25 and the lead portion 20 are to be removed from the body of the patient. Due to the removal sheath 60, when the electrode support 25 and the lead portion 20 are to be removed, the electrode support 25 can be prevented from passing through an opening of the blood vessel in an expanded state and damaging the vascular wall.

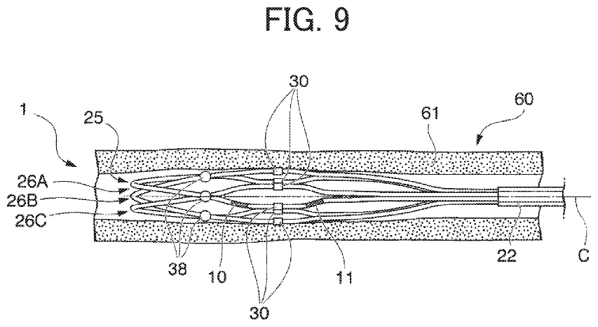

The removal sheath 60 is placed inside the blood vessel together with the electrode support 25 and the lead portion 20, during a period in which the electrode support 25 and the lead portion 20 are placed. The removal sheath 60 is provided with a sheath main body 61 (second cylindrical portion) and a hub 62 (second cylindrical portion), in this order from the tip end side. The sheath main body 61 is a cylindrical member through which the sheath main body 51 of the operation sheath 50 is inserted. An internal diameter of the sheath main body 61 is smaller than the external diameter of the electrode support 25 in the natural state, and greater than the smallest external diameter of the electrode support 25 in the contracted state. In a state in which the operation sheath 50 has been removed from the sheath main body 61, the lead portion 20 can be inserted into, and the contracted electrode FIG. 9 shows a state in which the electrode support 25 is held in the sheath main body 61. In the sheath main body 61, the elastic members 26A, 26B, 26C (six in total) with six projections 30 are packed. The internal diameter of the sheath main body 61 is required to be greater or equal to the external diameter of the sheath main body 51, and preferably determined relative to the external diameter of the sheath main body 51. For example in a case in which the external diameter of the sheath main body 51 is 2.0 mm, the internal diameter of the sheath main body 61 is required to be no less than 2.0 mm. The length of the sheath main body 61 is required. to be such that the entire electrode support 25 in the contracted state can be held, and a part of the base end side stays outside of the patient's body during placement. The length of the sheath main body 61 may be, for example, less than 50 mm and no greater than 100 mm.

The external diameter of the sheath main body 61 is not particularly limited as long as the sheath main body 61 can be inserted into a blood vessel and the body of a patient. The external diameter of the sheath main body 61 may vary in the longitudinal direction. In the present embodiment, a tapered portion 61a is provided on the outer periphery of a first end portion e1 close to the entire electrode support 25, the tapered portion 61a gradually decreasing in diameter in the longitudinal direction from a second end portion e2, which is on the opposite side of the first end portion e1, toward the first end portion e1. The external diameter of the tip end portion of the tapered portion 61a is preferably substantially the same as the external diameter of the sheath main body 51, so that a difference in level with the outer periphery of the sheath main body 51 is minimized. In the present embodiment, the external diameter of the sheath main body 61 more on the base end side than the tapered portion 61a is a predetermined diameter greater than that of the sheath main body 51. The removal sheath 60 is tapped into a neck region of a patient during use. Thickness of the sheath main body 61 may be increased on the base end side, which is to be positioned in the vicinity of the tapping position, in order to increase bend resistance and prevent bending in the vicinity the tapping position. The external diameter of the sheath main body 61 is preferably such that a thickness is no less than 0.1 mm. For example in a case in which the internal diameter of the sheath main body 61 is 2.0 mm, the external diameter of the sheath main body 61 is required to be no less than 2.2 mm. The external shape of the sheath main body 61 may be tapered from the base end side toward the tip end of the sheath main body 61.

The sheath main body 61 is required to be hard enough to bear external force applied from the electrode support 25 in the axial direction and the radial direction, when holding the electrode support 25. For example, the Shore hardness of the sheath main body 61 may be no less than 55D and no greater than 70D.

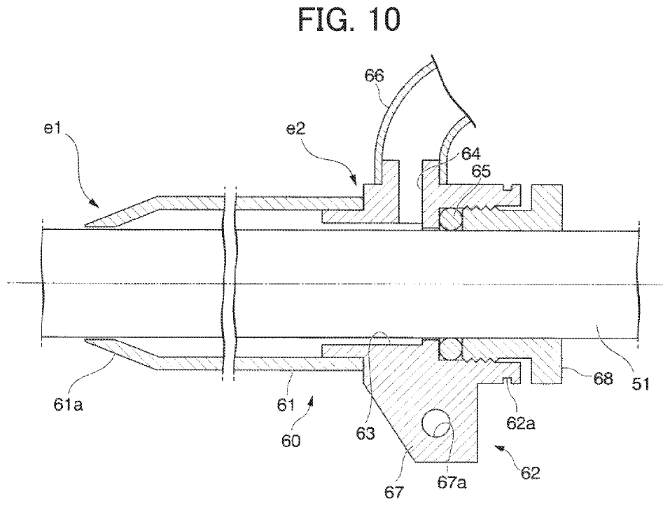

As shown in FIG. 10, the hub 62 is a substantially cylindrical member. The hub 62 is connected to the second end portion e2 of the sheath main body 61. The hub 62 is provided with a through-hole 63 that extends in the axis line direction and communicates with an inner space of the sheath main body 61, and a side hole 64 branched from the through-hole 63. A sealing member 65 (sealing portion, position fixing portion) such as an O-ring is provided to the through-hole 63. The internal diameter of the sealing member 65 is adjusted by a wind knob 68 (position fixing portion). The wind knob 68 is screwed into the hub 62. When the wind knob 68 is screwed into the hub 62, the sealing member 65 is compressed. The sealing member 65 is deformed through adjustment of a degree of compression thereof. As a result, the internal diameter of the sealing member 65 is adjusted. The internal diameter of the sealing member 65 can be changed from a diameter smaller than the external diameter of the lead main body 21 to a diameter greater than the external diameter of the sheath main body 51 of the operation sheath 50.

The minimum internal diameter of the sealing member 65 is defined to provide, when the sheath main body 51 is removed from the removal sheath 60, watertight sealing of a periphery of the lead main body 21 inserted through the operation sheath 60, whereby leakage or blood and the like can be prevented. The minimum internal diameter of the sealing member 65 in the present embodiment is defined to further obtain a frictional force for fixing a position of the lead main body 21 with respect to the removal sheath 60 in the longitudinal direction. The maximum internal diameter of the sealing member 65 is defined to allow the sheath main body 51 of the operation sheath 50, which is inserted into the removal sheath 60, to smoothly move in the longitudinal direction. As the internal diameter of the sealing member 65 is reduced by winding the wind knob 68, watertight sealing of a periphery the sheath main body 51 inserted through the operation sheath 60 is provided, whereby leakage of blood and the like can be prevented. The specific internal diameter of the sealing member 65 can be appropriately defined according to the elastic coefficient of the sealing member 65 and the frictional coefficient with respect to the lead main body 21 and the sheath main body 51.

The sealing member 65 and the wind knob 68 constitute the position fixing portion for fixing relative positions in the longitudinal direction with respect to the sheath main body 51 inserted into the sheath main body 61, and the lead portion 20 inserted into the sheath main body 61 when the operation sheath 50 has been removed.

A tube 66 is connected to the side hole 64. A connector 69 (see FIG. 1) such as a Luer-Lok connector is provided at the other end of the tube 66 (not illustrated). By connecting the connector 69 to a source of a medicinal solution such as physiological saline with heparin, the medicinal solution such as physiological saline with heparin can be supplied to the removal sheath 60 through the tube 66 and the side hole 64. It is to be noted that the tube 66 is not essential, and a connector or the like, to which an administration tube etc. can be connected, may be provided directly to the side hole 64.

The hub 62 is further provided with a vane portion 67 with a thread hole 67a, and the wind knob 68 provided on the base end side. The vane portion 67 can be used for fixing the removal sheath 60 on the body surface of a patient, by passing a thread through the patient's skin and the thread hole 67a. The thread passing through the skin may be wrapped around a thread groove 62a provided on the hub 62 as needed. By fixing the vane portion 67 on the patient's skin with a tape or the like, the removal sheath 60 can be prevented from rotating about the axis line during a treatment by the electrical stimulation electrode 1.

The sheath main body 61 and the hub 62 may be respectively formed from the same materials as the sheath main body 51 and the hub 52 of the operation sheath 50. As with the sheath main body 51, a metal blade may be enclosed in the sheath main body 61, and an appropriate coating film may be formed on the surface of the sheath main body 61. However, the removal sheath 60 is not required to be a peel-away sheath, since the removal sheath 60 is removed from the blood vessel together with the electrode support 25 and the lead portion 20 after the indwelling of the electrode support 25.

Hereinafter, the electrical stimulation device 70 is described. The electrical stimulation device 70 is formed in size and weight allowing a patient to carry. The electrical stimulation device 70 is provided with an operation panel on which an operator operates. Various types of operation buttons (not illustrated), and the notification member 75 are provided on the operation panel. A battery-driven electrical circuit board is installed in the electrical stimulation device 70. Wirings from the lead portion 20 are connected to the electrical circuit board.

FIG. 11A is a block diagram schematically showing an electrical circuit and the like provided in the electrical stimulation device 70. The electrical stimulation device 70 includes: the stimulus output generating circuit 72 that generates electric energy to be output to the stimulation electrode portions 10, 11; the heart rate detection circuit 73 that detects a heart rate on the basis of the heartbeat information detected by the heartbeat detection electrodes 15, 16; the heartbeat status determination circuit 74 that determines a heartbeat status on the basis of the heart rate detected by the heart rate detection circuit 73; and the notification member 75 that notifies the heartbeat status determined by the heartbeat status determination circuit 74. The stimulus output generating circuit 72 is connected to the stimulation electrode portions 10, 11 via the wirings 35 as the conductor. The heart rate detection circuit 73 is connected to the heartbeat detection electrodes 15, 16 via the wirings as the conductor. The heart rate detection circuit 73 is electrically connected to the heartbeat status determination circuit 74. It is to be noted that the stimulus output generating circuit 72, the heart rate detection circuit 73, and the heartbeat status determination circuit 74 are not necessarily separated physically and may be configured integrally as a circuit (for example, processor). The stimulus output generating circuit 72, the heart rate detection circuit 73, and the heartbeat status determination circuit 74 constitute an operation unit 71 of the electrical stimulation device 70. The operation unit 71 including the stimulus output generating circuit 72, the heart rate detection circuit 73 and the heartbeat status determination circuit 74 performs the processes described below.

FIGS. 11B, 11C, and 11D are schematic views showing examples of an electric stimulating pulse as the electric energy generated by the stimulus output generating circuit 72 of the electrical stimulation device 70. In FIGS. 11B, 11C, and 11D, an ordinate represents electric current or voltage, and an abscissa represents time t. As shown in FIG. 11B, the stimulus output generating circuit 72 generates, for example, a stimulating pulse which is in a biphasic waveform of the constant-current system or the constant-voltage system. However, the stimulating pulse generated by the stimulus output generating circuit 72 is not limited to the biphasic waveform. Intensity of the stimulating pulse can be changed digitally. For example, the intensity of the electric current can be changed stepwisely by 0.5 mA in a range of 0 to 20 mA. The stimulus output generating circuit 72 generates, for example, the stimulating pulse with a frequency of no less than 10 Hz and no greater than 20 Hz (pulse cycle T1 being no less than 0.05 sec and no greater than 0.1 sec), and a pulse width of no less than 50 μsec and no greater than 400 μsec.

Since the stimulating pulse is in the biphasic waveform, in a positive waveform part, a point stimulating the vagus nerve is at the negative electrode position. The subsequent pulse is in a negative waveform part. In the negative waveform part, a point stimulating the vagus nerve is at the positive electrode position. Through application of the biphasic waveform, the vagus nerve can be stimulated at both electrode positions of the stimulation electrode portions 10, 11, and thus a stimulated range is large. As a result, the operator can easily find a position to stimulate. The stimulating pulse is transmitted to the stimulation electrode portions 10, 11 via the wirings 35 in the lead portion 20.

The stimulus output generating circuit 72 of the electrical stimulation device 70 is capable of outputting the stimulating pulse as the electric energy generated in a plurality or output modes. The plurality of output modes includes: a continuous stimulation mode in which the stimulating pulse is continuously output; and an intermittent stimulation mode in which a stimulating pulse group is intermittently output. The plurality of output modes is selectively switchable by the operator operating on the operation panel as an input unit.

FIG. 11C shows a pulse shape of the continuous stimulation mode. In the continuous stimulation mode, the stimulating pulse is continuously output for a predetermined time period (application period Ton). The output is started and stopped by the operator operating the electrical stimulation device 70. It is desirable that the stimulation electrode portions 10, 11 are opposed to the vagus nerve, in order to efficiently transmit the electric energy to the vagus nerve outside of the blood vessel. The opposite positional relationship is determined through a decrease in heart rate. The operator can more easily detect the decrease in heart rate in the continuous stimulation mode. Since the operator may forget to stop the output, a configuration of automatically stopping the output after a predetermined period of time may further be employed.

FIG. 11D shows a pulse shape of the intermittent stimulation mode. In the intermittent stimulation mode, a stimulating pulse group constituted of a plurality of stimulating pulses is applied repeatedly for the application period Ton, in an application cycle T2. For example, the application period Ton may be no less than 3 sec and no greater than 20 sec, and the application cycle T2 may be 30 sec or 60 sec. The application cycle T2 may also be an arbitrary cycle of no less than 30 sec. The stimulation in the intermittent stimulation mode is to be carried out for several days as a treatment period. For example, it has been known that, through stimulation of the vagus nerve on the back side of the superior vena cava after the reperfusion treatment for an acute myocardial infarction patient, a decrease in heart rate is expected to alleviate heart strain and provide an anti-inflammatory effect, thereby preventing an infarct area from growing. In the case of such a treatment, the intermittent stimulation. mode imposes less burden on a patient and is therefore more preferred.

As described above, the nerve stimulation apparatus 90 of the present embodiment preferably outputs the stimulating pulse in the continuous stimulation mode during positioning of the stimulation electrode portions 10, 11 as the intravascular stimulation electrodes, and outputs the stimulating pulse in the intermittent stimulation mode during a treatment period after the positioning of the stimulation electrode portions 10, 11.

The heart rate detection circuit 73 detects a heart rate on the basis of the heartbeat information detected by the heartbeat detection electrodes 15, 16. FIG. 11E is a schematic view showing heart rate variation detected by the heart rate detection circuit 73 while the stimulus output generating circuit 72 outputs the stimulating pulse in the intermittent stimulation mode. In FIG. 11E, an ordinate represents the heart rate HR, and an abscissa represents time t. The heart rate decreases in sync with the application period Ton. The heart rate starts to decrease immediately after the application of the stimulus output, and after several seconds, the minimum heart rate is reached. The minimum heart rate is then maintained during the application period Ton. Thereafter, the heart rate is restored to the original rate gradually after the stop of stimulus output. For example, in the case of the application period Ton being 10 sec and the application cycle T2 being 60 sec in the intermittent stimulation mode, a decrease in heart rate is observed every 60 seconds. The time required to reach the minimum heart rate is about 3 sec from the start of application of the stimulus.

The heartbeat status determination circuit 74 determines a heartbeat status on the basis of the heart rate detected by the heart rate detection circuit 73. The heartbeat status determination circuit 74 memorizes and compares heart rates of different timing, and calculates a heart rate reduction rate as the heartbeat status. More specifically, the heartbeat status determination circuit 74 calculates a heart rate reduction rate on the basis of a pre-stimulation heart rate HR_base detected by the heart rate detection circuit 73 before the stimulation output generating circuit 72 starts outputting the electric energy, and an in-stimulation heart rate HR_st detected by the heart rate detection circuit 73 while the stimulation output generating circuit 72 outputs the electric energy. As used herein, the heart rate reduction rate means a reduction level of the in-stimulation heart rate HR_st with respect to the pre-stimulation heart rate HR_base. For example, the heart rate reduction rate is represented by the following equation (1).

heart rate reduction rate (%)=((HR_base)−(HR_st))/(HR_base)) ×100 . . . (1)

After the reperfusion treatment for an acute myocardial infarction patient, a drop in blood pressure of the patient is the most feared symptom. During the stimulation of the vagus nerve on the back side of the superior vena cava, stimulation intensity is required to be such that the patient's blood pressure is not affected. When the heart rate reduction rate is greater than 20%, the blood pressure may drop. It is therefore suitable to adjust the stimulation intensity such that the heart rate reduction rate is no greater than 20%. More specifically, it is suitable that the stimulation intensity of the electrical stimulation device 70 is adjusted such that the heart rate reduction rate is no less than 5% and no greater than 10%. The output for electrically stimulating the vagus nerve (current value, voltage value, pulse width, application cycle, and the like) may be controlled to obtain. such a stimulation intensity. In the present embodiment, the electrical stimulation device 70 is provided with the following safety functions.

The heartbeat status determination circuit 74 makes the notification member 75 notify the heartbeat status in a case in which at least the heart rate reduction rate is less than a first threshold value. The heartbeat status determination circuit 74 stops the stimulation output generating circuit 72 from outputting the electric energy in a case in which the heart rate reduction rate is no less than the first threshold value. The first threshold value is for example no less than 15% and no greater than 25%, preferably no less than 18% and no greater than 22%, and more preferably 20%.

As the notification member 75, a notification member emitting light (LED, lamp, etc.), a notification member producing sound (buzzer, speaker, etc.) or the like is used. For example, the heartbeat status determination circuit 74 activates the notification member 75 to notify the heartbeat status in a case in which the heart rate reduction rate is less than 20% (first threshold value). In the case of using a plurality of LEDs as the notification member 75, changing color of the LEDs according to the heart rate reduction rate can improve visual recognizability for the operator. For example, when the heart rate reduction rate is less than 5%, an orange LED is turned on; when the heart rate reduction rate is no less than 5% and less than 10%, a green LED is turned on; and when the heart rate reduction rate is no less than 10%, a red LED is turned on. The notification member 75 is thus provided with a plurality of LEDs of different colors; and the heartbeat status determination circuit 74 turns on one of the plurality of LEDs of a color corresponding to the heart rate reduction rate.

Here, a state notified by the orange LED is a first notification state indicating that the stimulated position is inappropriate or stimulation intensity is insufficient. A state notified by the green LED is a second notification state indicating that the stimulation intensity is appropriate. A state notified by the red LED is a third notification state indicating that the heart rate reduction rate is too high, i.e., in a warning state. The notification member 75 can notify, in accordance with the heart rate reduction rate, in an ascending order of the heart rate reduction rate: a first notification state indicating that an intensity of stimulation of the vagus nerve is insufficient; a second notification state indicating that the intensity of stimulation of the vagus nerve is appropriate; and a third notification state indicating that the heart rate reduction rate is in the warning state.

In. other words, in a case in which the heart rate reduction rate is no less than the second threshold value (for example, 10%) smaller than the first threshold value and less than the first threshold value (for example, 20%), the heartbeat status determination circuit 74 makes the notification member 75 notify in the third notification state indicating that the heart rate reduction rate is in the warning state. Meanwhile, in a case in which the heart rate reduction rate is no less than the third threshold value (for example, 5%) smaller than the second threshold value and less than the second threshold value (for example, 10%), the heartbeat status determination circuit 74 makes the notification member 75 notify in the second notification state indicating that the intensity of stimulation of the vagus nerve is appropriate. Furthermore, in a case in which the heart rate reduction rate is less than the third threshold value (for example, 5%), the heartbeat status determination circuit 74 makes the notification member 75 notify in the first notification state indicating that the intensity of stimulation of the vagus nerve is insufficient. It is to be noted that the second threshold value is preferably, for example, no less than 8% and no greater than 15%. The third threshold value is preferably, for example, no less than 2% and no greater than 8%. A difference between the first threshold value and the second threshold value is preferably no less than 5%. A difference between the second threshold value and the third threshold value is preferably no less than 3%.

It is to be noted that, when the heart rate reduction rate is in the third notification state (warning state), the third notification state (warning state) is maintained until the heart rate reduction rate is improved. The notification member 75 is capable of notifying the warning state, and thus also serves as a waning member.

The heartbeat status determination circuit 74 automatically stops the stimulation output generating circuit 72 from outputting the electric energy in a case in which the heart rate reduction rate is no less than 20% (first threshold value), out of concern of a drop in blood pressure. In this case, the notification member 75 may either maintain the third notification state (warning state), or, for example, blink the red LED as a fourth notification state indicating that the electric energy output has been automatically stopped. Otherwise, the notification member 75 may stop working.

It is to be noted that, as described above with reference to FIG. 11E, the heart rate starts to decrease immediately after the application of the stimulus, and after severest seconds, the minimum heart rate is reached. The minimum heart rate is then maintained during the application period Ton. Thereafter, the heart rate is restored to the original rate gradually after the stop of stimulus output. Here, when the heart rate reduction rate is being calculated, the in-stimulation heart rate HR_st can also be obtained by averaging the heart rates during the application period Ton. However, in order to obtain the heart rate reduction rate due to the stimulation more accurately, it is more preferred to obtain the in-stimulation heart rate HR_st by, for example, averaging the heart rates detected after a lapse of a determination stand-by period Tw since the stimulus output generating circuit 72 starts outputting the electric energy, until the stimulus output generating circuit 72 stops outputting the electric energy. For example, since it takes about 3 sec after starting application of stimulus to reach the minimum heart rate, the in-stimulation heart rate HR_st is determined by using the heart rates obtained until the end of the application period Ton, with the determination stand-by period Tw being about 3 sec. As described above, the determination stand-by period Tw is preferably 3 sec, or no less than 3 sec.

The heartbeat status determination circuit 74 thus calculates the heart rate reduction rate on the basis of the pre-stimulation heart rate and the in-stimulation heart rate, and then activates the notification member 75. In addition, the heartbeat status determination circuit 74 automatically stops the stimulation output generating circuit 72 from outputting the electric energy in a case in which the heart rate reduction rate is no less than 20%. In this case, the stimulation output generating circuit 72 and the heart rate detection circuit 73 are in sync, and a pre-stimulation period and an in-stimulation period are distinguished. The heartbeat status determination circuit 74 thus can calculate the heart rate reduction rate through comparison between the pre-stimulation heart rate HR base and the in-stimulation heart rate HR_st. The pre-stimulation heart rate and the in-stimulation heart rate are stored in a storage unit of the heartbeat status determination circuit 74 and are used for calculation of the heart rate reduction rate.

The heart rate is reduced by stimulating the vagus nerve as described above; however, an appropriate reduction of the heart rate is required for a treatment. More specifically, since the heart rate reduction rate of no less than 20% may cause a drop in blood pressure, it is desirable that notification member 75 is activated to warn the operator when the heart rate reduction rate is no less than 10%, and the stimulation output from the nerve stimulation apparatus is stopped when the heart rate reduction rate is no less than 20%.

It is to be noted that, in order to improve safety, it is preferred to analyze only the pre-stimulation heart rate HR base prior to starting stimulation for the application period Ton and, if bradycardia or tachycardia is detected, to activate the notification member 75 or to control not to start the stimulation output.

Safety can be further improved by installing a semiconductor pressure sensor in the lead portion 20 for measuring blood pressure. The semiconductor pressure sensor may be installed anywhere in contact with blood in a blood vessel. The electrical stimulation device 70 is further provided with a blood pressure detection circuit and a blood pressure determination circuit. The blood pressure determination circuit determines whether pre-stimulation blood pressure is within a predetermined range, and whether a stimulus can be given. Furthermore, the blood pressure determination circuit may compare the pre-stimulation blood pressure with in-stimulation blood pressure, determines based on the blood pressure reduction ratio etc., and activate the notification member 75 or automatically stop the stimulation output from the stimulation output generating circuit 72. In this case, if the stimulation output is appropriate, i.e., the heart rate reduction rate is between 5 to 10%, blood pressure does not drop. Since the heart rate reduction rate greater than 20% may cause a drop in blood pressure, detection and determination of the blood pressure can improve safety. In addition, the pre-stimulation blood pressure is already low, it is possible not to start the stimulation.

In general, since a patient's heart rate changes according to his/her clinical condition, it is considered to be difficult to obtain an optimal value by just controlling the output of electric stimulation of the vagus nerve. An easily-detectable index that changes in response to the electrical stimulation of the vagus nerve is exemplified by a heart rate and blood pressure. By detecting these indices, an optimal stimulation output can be obtained and safety of the patient can further be improved.

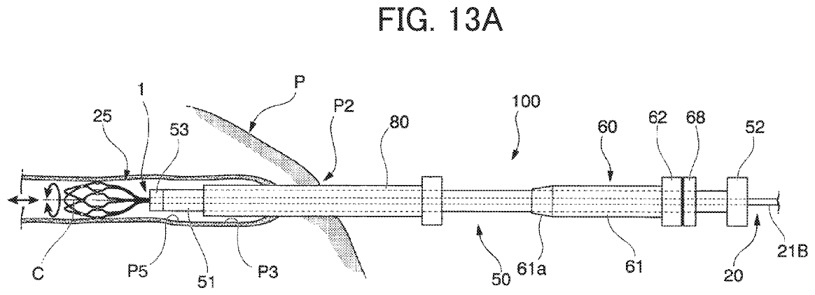

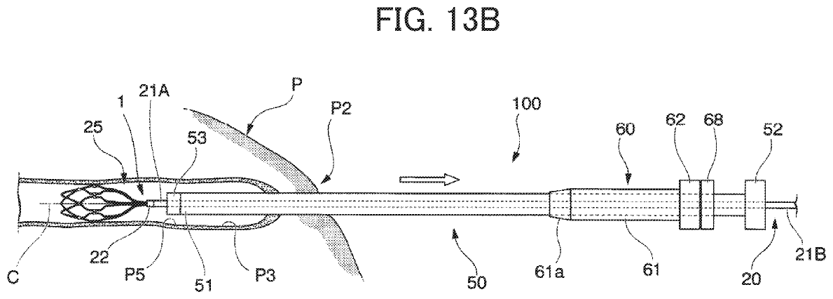

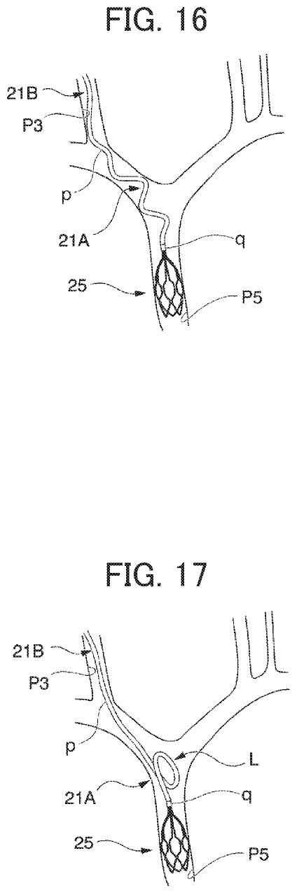

Hereinafter, functions of the medical instrument set 100 are described along with a placement operation and a removal operation of the electrical stimulation electrode 1. FIG. 12 is a schematic view showing a state in which the biasing member of the medical instrument set according to the embodiment of the present invention is inserted into the superior vena cava. FIGS. 13A, 13B, 14A, 14B and 15 are schematic views showing a placement operation of a medical instrument in the medical instrument set according to the embodiment of the present invention. FIGS. 16 and 17 are schematic views showing an example of slack of the linear body of the medical instrument set according to an embodiment of the present invention. In FIGS. 12 to 17, the stimulation electrode portions 10, 11 and the heartbeat detection electrodes 15, 16 are omitted.

As shown in FIG. 1, in the medical instrument set 100, the operation sheath 50 is moved toward the tip end side, and the engaging portion 53 of the operation sheath 50 is engaged with the electrode support fixation member 22. When the engaging portion 53 is disengaged from the electrode support fixation member 22, the operator engages the engaging portion 53 with the electrode support fixation member 22. Prior to use of the medical instrument set 100, the operator fills the operation sheath 50 and the removal sheath 60 with physiological saline with heparin or the like supplied respectively from the tubes 56, 66. As a result, air is removed from the operation sheath 50 and the removal sheath 60 prior to use. The operator attaches to a patient an electrocardiograph for measuring a heart rate. As shown in FIG. 12, the operator makes a small incision to form an opening P2 in the vicinity of a neck P1 of a patient P. FIG. 12 shows a state after placement of the electrode support 25, and therefore the operation sheath 50 and an introducer 80 are omitted. As shown in FIG. 13A, he operator attaches the introducer 80 to the opening P2. The introducer 80 is a tubular member which has such an internal diameter that the sheath main body 51 of the operation sheath 50 can be inserted therethrough, and is shaped to be insertable up to the superior vena cava P5. In the present embodiment, it is not required to insert the removal sheath 60 into the introducer 80.

The introducer 80 is attached such that the tip end portion thereof reaches the superior vena cava P5 (blood vessel), where the electrode support 25 is to be placed, via the right vena jugularis interna P3. The base end portion of the introducer 80 protrudes outward from the opening P2 of the patient P. The introducer 80 is removed from the blood vessel after completion of placement of the electrode support 25 described later. In this case, the introducer 80 may be removed by using a surgical instrument such as scissors, or the introducer 80 may be configured as a peel-away sheath that can be removed hand. The peel-away configuration is exemplified by the configuration for the operation sheath 50.

As shown in FIG. 13A, the operator inserts the electrode support 25 and the operation sheath 50 into the right vena jugularis interna P3 (blood vessel) via the introducer 80. Prior to insertion, the electrode support 25 is elastically deformed (contracted) to have the external diameter insertable into the introducer 80. The electrode support 25 and the operation sheath 50 are inserted to slide against an inner peripheral surface of the introducer 80. The removal sheath 60 is arranged at an appropriate position on the sheath main body 51 outside of the introducer 80. The removal sheath 60 can be positioned on the sheath main body 51 by winding the wind knob 68 to avoid displacement on the sheath main body 51. Upon insertion of the electrode support 25, the operator checks positions of the stimulation electrode portions 10, 11 of the electrical stimulation electrode 1, the wire portion 33 of the electrode support 25, and the like under radioscopy.

The operator moves the electrode support 25 to the tip end portion of the introducer 80 by advancing the operation sheath 50 and the lead portion 20. As used herein, “advancing” means an action of pushing the operation sheath 50 and the lead portion 20 toward the tip end side (distal end side). An action of bringing back the operation sheath 50 and the lead portion 20 toward the base end side (proximal end side) is called “retracting”.

Next, the operator moves the electrode support 25 and the operation sheath 50 relative to the introducer 80, to discharge the electrode support 25 from the introducer 80. The electrode support 25 is introduced into the superior vena cava P5 on a front side of the introducer 80 (see FIG. 13A). Upon discharging the electrode support 25, the operator may advance the operation sheath 50 while fixing the position of the introducer 80. Alternatively, upon discharging the electrode support 25, the operator may retract the introducer 80 while fixing the positions of the electrode support 25 and the operation sheath 50.

Once the electrode support 25 is discharged from the introducer 80, the contracted electrode support 25 expands with an elastic restoring force. Since the external diameter of the electrode support 25 in the natural state is greater than the internal diameter of the superior vena cava P5, the electrode support 25 pushes against the inner wall of the superior vena cava P5. The stimulation electrode portions 10, 11 (not illustrated in FIG. 13A) are exposed outward in a radial direction of the electrode support 25 and are therefore in contact with the inner wall of the superior vena cava P5. The electrode support 25 is thus placed in the superior vena cava P5. After the introduction of the electrode support 25 into the superior vena cava P5, the operator retracts the introducer 80 by about 50 mm to 100 mm with respect to the electrode support 25, in order to facilitate the subsequent manipulation.

As an example, in the present embodiment, the electrode support 25 is introduced into the superior vena cava P5 via the right vena jugular's interna P3 as described in the foregoing. However, the electrode support 25 may also be introduced into the superior vena cava P5 via, in addition to the right vena jugularis interns P3, the right vena jugularis externa, the left vena jugular's externa, the left vena jugularis interna, the right subclavian vein, the left suhclavian vein, and the like.

As shown in FIG. 12, the vagus nerve P6 (nerve) to be stimulated is found in parallel to, and adjacent to, the superior vena cava P5. The operator positions the electrode support 25 such that the stimulation electrode portions 10, 11 (not illustrated in FIG. 12) of the electrode support 25 can stimulate the vagus nerve P6. The operator operates the electrical stimulation device 70 to apply the stimulating pulse to the stimulation electrode portions 10, 11 in the continuous stimulation mode. The stimulating pulse is transmitted to the inner wall or the blood vessel.

In this state, when the operator advances or retracts the operation sheath 50 and the lead portion 20 while holding the base end portions thereof, the electrode support 25 on the tip end side moves along the axis line direction of the superior vena cava P5. When the operator rotates the operation sheath 50 about the axis line while holding the base end portions of the operation sheath 50 and the lead portion 20, the engaging portion 53 on the tip end portion of the operation sheath 50 rotates in the same direction. As a result, the electrode support fixation member 22 engaged with the engaging port ton 53, the lead portion 20, and the electrode support 25 also rotate in the same direction. By thus advancing, retracting, and rotating the operation sheath 50 and the lead portion 20 while holding the base end portions thereof, the operator can adjust the position of the electrode support 25 in the axis line direction and the circumferential direction in the superior vena cava P5. The operator adjusts the position while measuring the heart rate with the electrocardiograph or the like attached to the patient P. When the stimulation electrode portions 10, 11 get closer to, and are arranged to face, the vagus nerve P6, the electric stimulation applied from the stimulation electrode portions 10, 11 to the vagus nerve P6 increases, and the heart rate of the patient P is the lowest. The operator adjusts the position of the electrode support 25 such that the heart rate is the lowest. At the same time, the heart rate detection circuit 73 of the electrical stimulation device 70 starts detecting the heart rate on the basis of signals input from the heartbeat detection electrodes 15, 16 that detect the heartbeat information in the blood vessel. And the heartbeat status determination circuit 74 starts determining the heartbeat status. The heartbeat status determination circuit 74 calculates the heart rate reduction rate on the basis of the pre-stimulation heart rate and the in-stimulation heart rate, and makes the notification member 75 notify the heartbeat status on the basis of the heart rate reduction rate. The heartbeat status determination circuit 74 automatically stops the stimulation output generating circuit 72 from outputting the electric energy in a case in which the heart rate reduction rate is greater than 20%.

After the positioning of the electrode support 25, the operator turns off the continuous stimulation mode and places the electrode support 25. The operator fixes the position of the lead portion 20 in the axis line direction while holding the base end portion of the lead portion 20. In this case, the operator 50 retracts the operation sheath 50. Since the electrode support 25 is pressed against the inner wall of the superior vena cava P5 to be frictionally engaged therewith, the engaging portion 53 of the operation sheath 50 is retracted along the axis line C. The engaging portion 53 is thus detached from the electrode support fixation member 22. After retracting the operation sheath 50 until at least a part of the sot portion 21A of the lead main body 21 is exposed on a front side of the operation sheath 50, the operator stops moving the operation sheath 50. In the present embodiment, an amount of retraction of the operation sheath 50 is preferably 100 mm, for example. In this way, when the engaging portion 53 is detached from the electrode support fixation member 22, the electrode support 25 is connected to the operation sheath 50 through the lead main body 21 therebetween. The soft portion 21A is provided on the tip end portion of the lead main body 21. Therefore, even if the tip end portion of the operation sheath 50 displaces, or the external force is transmitted from the operation sheath 50 to the non-soft portion 21B, the external force caused in these cases is absorbed by the soft portion 21A. As a result, the removal operation of the introducer 80 (described later) does not displace the electrode support 25.

In the foregoing description of the operations, the case of retracting only the operation sheath 50 has been explained. However, the operator may also advance the lead portion 20 while retracting the operation sheath 50. In this case, since the soft portion 21A is provided on the tip end side of the lead portion 20, the soft portion 21A slacks when the non-soft portion 21B on the base end portion is advanced. As a result, the electrode support 25 is not displaced. In this case, since the slack of the soft portion 21A increases as the lead portion 20 advances, displacement of the electrode support 25 can be prevented more reliably.

Next, the operator removes the introducer 80 from the body of the patient P. For example, the operator pulls out the introducer 80 from the body by retracting the introducer 80. Furthermore, the operator tears the introducer 80 outside the body from the base end side. For tearing the introducer 80, the operator may use a surgical instrument such as scissors. In the case in which the introducer 80 is configured to be peelable, the operator may tear the introducer 80 by hand. As described above, the introducer 80 is removed as shown in FIG. 13B. The operation sheath 50 is inserted into the opening P2.

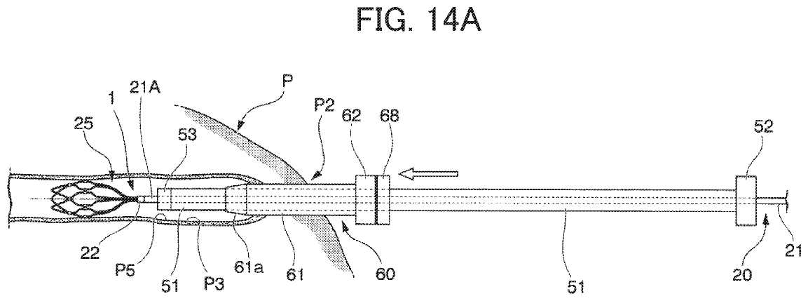

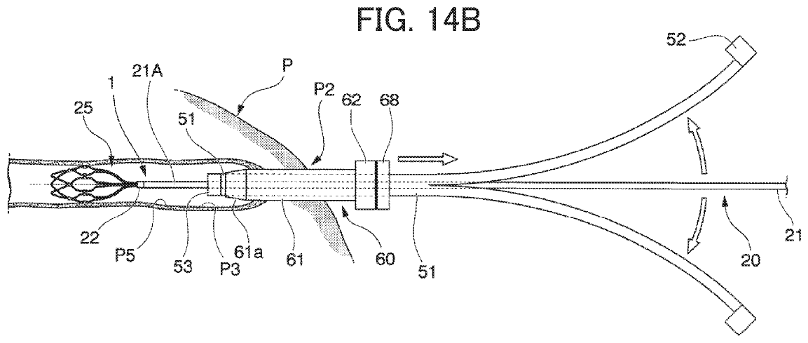

After the removal of the introducer 80, the operator advances the removal sheath 60 along the sheath main bod 51 as shown in FIG. 14A. In the case in which the removal sheath 60 is fixed on the sheath main body 51, the operator advances the removal sheath 60 after unfixing it by unwinding the wind knob 68 or the like. While the removal sheath 60 is outside the body, sealing of a gap with the sheath main body 51 is not necessary, and thus the removal sheath 60 can be quickly and easily moved by unwinding the wind knob 68 appropriately.

Once the tip end portion of the removal sheath 60 reaches the opening P2, the operator winds the wind knob 68 to such a degree that the removal sheath 60 can slide with respect to the sheath main body 51. As a result, the sealing member 65 (see FIG. 10) is in close contact with the outer peripheral surface of the sheath main body 51, to thereby seal a gap between the hub 62 and the sheath main body 51.

The operator inserts the tip end portion of the removal sheath 60 into the opening P2 as shown in FIG. 14A. In this case, the removal sheath 60 advances while being guided by the sheath main body 51. Therefore, the removal sheath 60 is inserted accurately into a gap between the opening P2 and the outer peripheral surface of the sheath main body 51, at the opening P2 into which the sheath main body 51 is inserted. In the present embodiment, the tapered portion 61a provided on the tip end portion of the removal sheath 60 allows the tip end portion of the removal sheath 60 to move into the opening P2 smoothly, without being caught. The opening P2 gradually increases in diameter along the outer peripheral surface of the tapered portion 61a. As a result, damage of the opening P2 and a blood vessel such as the right vena jugularis interna P3, and enlargement of a wound can be prevented during insertion of the removal sheath 60. Once the removal sheath 60 is inserted, the opening P2 is in close contact with the outer peripheral surface of the sheath main body 1. In the case of such an insertion operation of the removal sheath 60, the operator is only required to insert the removal sheath 60 while using the sheath main body 51 as a guide. The operator can thus easily insert the tip end portion of the removal sheath 60 into the opening P2. In this case, since the removal sheath 60 does not cause damage or a blood vessel and enlargement of a wound during the insertion operation, the operator can carry out the insertion operation at ease.

Once it is confirmed that the tip end portion of the removal sheath 60 has reached the inside of the right vena jugularis interna P3, the operator fixes the position of the removal sheath 60. The removal sheath 60 may be fixed by, for example, passing a thread through the patient's skin and the thread hole 67a (see FIG. 10), and wrapping around the thread groove 62a (see FIG. 10 as needed. Alternatively, the removal sheath 60 may be fixed by, for example, fixing the vane portion 67 (see FIG. 10) on the patient's skin with a tape or the like. By fixing the removal sheath 60 onto the patient's body surface, the removal sheath 60 can be reliably maintained in a state of being inserted into the right vena jugularis interna P3.

Next, the operator confirms, through changes in heart rate and the like, that the position of placement of the electrode support 25 has not been changed, and then removes the operation sheath 50. If the position of placement of the electrode support 25 has been changed due to the insertion operation of the removal sheath 60, the body motion of the patient P or the like, the operator readjusts the position placement of the electrode support 25.

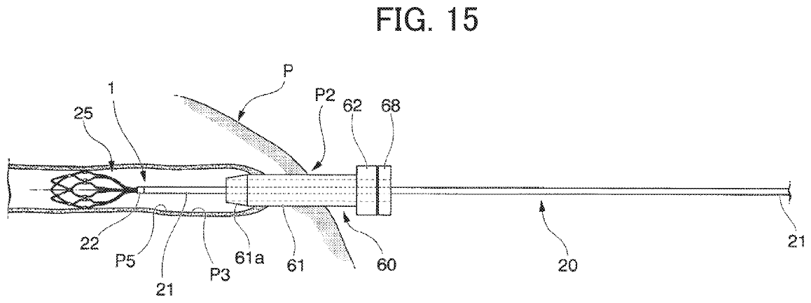

In order to remove the operation sheath 50, the operator retracts the operation she 50 as shown in FIG. 14B. Furthermore, the operator tears the operation sheath 50 outside the body from the base end side. For tearing the operation sheath 50, the operator may use a surgical instrument such as scissors. In the case in which the introducer 80 is configured to be peelable, the operator may tear the introducer 80 my hand. After pulling out the distal end of the operation sheath 50 from the removal sheath 60, the operator winds the wind knob 68 to seal a gap with the lead main body 21 of the lead portion 20. As described above, the operation sheath 50 is removed as shown in FIG. 15.

After removing the operation sheath 50, the operator may also advance and introduce the lead portion 20 into the superior vena cava P5, so as to further increase the length of the lead portion 20 staying in the patient's blood vessel. When the lead portion 20 is advanced, the slack of the soft portion 21A in the patient's blood vessel increases. Due to the slack of the soft portion 21A, for example, even when the lead portion 20 on the base end side moved by body motion of the patient P, the movement of the lead port on 20 on the base end side is not transmitted to the electrode support 25. As a result, the placement position of the electrode support 25 is stabilized.

shape of the slack of the soft portion 21A is not particularly limited. It is because the soft portion 21A is only required to have slack in order to absorb the external force acting on the lead portion 20 on the base end side before transmission thereof to the electrode support 25. For example, as shown in FIG. 16, the soft portion 21A can have meandering slack in the blood vessel between a point p and a point q. For example, as shown in FIG. 17, the soft portion 21A can have a loop part L between the point p and the point q. In every case, an actual length of the soft portion 21A between the point p and the point q is greater than the minimum length along the blood vessel between the point p and the point q.

The shape and amount of the slack can be checked by, for example, observing the shape of the lead portion 20 under radioscopy. The amount of the slack can be determined to absorb possible displacement of the lead portion 20 in the base end portion inside the body of the patient P.

Once the amount of the slack of the soft portion 21A is adjusted, the placement of the electrode support 25 is completed. The operator winds the wind knob 68 to such a degree that the lead main body 21 cannot move in the longitudinal direction. As a result, the lead portion 20 is positioned with respect to the removal sheath 60.

The timing to adjust the amount of the slack of the soft portion 21A is not particularly limited as long as it is prior to the completion of the placement. For example, after disengaging the engaging portion 53 of the operation sheath 50 from the electrode support fixation member 22 in the superior vena cava P5, the amount of the slack of the soft portion 21A can be adjusted. while the operation sheath 50 stays in the blood vessel.

When it is no longer needed to move the lead portion 20, for example upon completion of adjustment of the amount of the slack of the soft portion 21A carried out as needed, the operator winds the wind knob 68 to such a degree that the lead portion 20 cannot move in the longitudinal direction. For example, in the case in which the adjustment of the amount of the slack has been completed while the operation sheath 50 is inserted into the blood vessel, the operator may wind the wind knob 68 to such a degree that the lead portion 20 cannot move in the longitudinal direction, when the operation sheath 50 is pulled out from the removal sheath 60.

As described in the foregoing, once the removal of the operation sheath 50 is completed and the position of the lead portion 20 is fixed, the placement of the electrode support 25 is completed.

Since the tube 66 is connected to the hub 62 of the removal sheath 60, the operator can also administer liquid such as physiological saline with heparin, by connecting a syringe pump or the like to the connector 69. For example, in a case of administering an anticoagulant, the anticoagulant thus discharged is diffused in the vicinity of the lead portion 20 and the electrode support 25 through the bloodstream, and can reduce generation of a thrombus at positions were the lead portion 20 and the electrode support 25 are placed. The electrode support 25 and a part of the lead portion 20 are thus placed in the blood. vessel of the patient P.

The stimulus output generating circuit 72 of the electrical stimulation device 70 applies the stimulating pulse to the stimulation electrode portions 10, 11 of the electrode support 25 placed in the superior vena cava P5. Here, by applying the stimulating pulse in the intermittent stimulation mode such that the heart rate reduction rate is 5 to 10%, a nerve stimulation treatment can be provided to the patient P. At the same time, the heart rate detection circuit 73 of the electrical stimulation device 70 starts detecting the heart rate on the basis of signals input from the heartbeat detection electrodes 15, 16 that obtain the heartbeat information in the blood vessel. And the heartbeat status determination circuit 74 starts determining the heartbeat status. The heartbeat status determination circuit 74 calculates the heart rate reduction rate on the basis of the pre-stimulation heart rate and the in-stimulation heart rate, and makes the notification member 75 notify the heartbeat status on the basis of the heart rate reduction rate. The heartbeat status determination circuit 74 automatically stops the stimulation output generating circuit 72 from outputting the electric energy in a case in which the heart rate reduction rate is greater than 20%.

The heartbeat detection electrodes 15, 16 as the intravascular heartbeat detection electrode are provided In the lead portion 20 of the electrical stimulation electrode 1 placed in the blood vessel and detects the heartbeat information in the blood vessel. Therefore, during the treatment period of several days, the heartbeat information can be detected constantly without attaching an electrocardiograph to the patient's body surface. As a result, consideration of arrangement of a lead of an electrocardiograph on the patient's body surface is no longer required, and, during the treatment period of several days, safety can be improved through activating the notification member, stopping output of the electric energy, etc. on the basis of the heartbeat information obtained inside the blood vessel.

After completion of the nerve stimulation treatment required for the patient P, the electrical stimulation electrode 1 is removed as described below. In the present embodiment, no re-operation is required for removing the electrical stimulation electrode 1. The operator unwinds the wind knob 68 of the removal sheath 60 to enable the lead portion 20 to slide. In this state, the operator holds and pulls the lead portion 20 to retract the electrode support 25. Alternatively, the operator may also hold the hub 62 of the removal sheath 60 in order to reliably prevent disengagement of the removal sheath 60 from the right vena jugularis interna P3.

As the lead portion 20 is pulled toward the base end side, the electrode support 25 is retracted along the blood vessel. Here, since the electrode support 25 has a basket-like shape with the base end side gradually reducing in diameter, the electrode support 25 can be retracted smoothly. Once the electrode support 25 is retracted to the tip end portion of the removal sheath 60, the operator retracts further the electrode support 25. As a result, the electrode support 25 is pulled into the sheath main body 61 from the base end side. The electrode support 25 contracts along the inner peripheral face of the sheath main body 61, and is thus stored in the removal sheath 60.

Once the electrode support 25 is stored in the sheath main body 61, the operator detaches the hub 62 of the removal sheath 60 from the skin of the patient P by, for example, cutting a thread. This detachment operation may be carried out before unwinding the wind knob 68 of the removal sheath 60. Thereafter, the operator pulls out the removal sheath 60 from the right vena jugularis interna P3, through the opening P2. In this way, only the sheath main body 61 having a predetermined external diameter passes through the opening P2. The electrode support 25 can thus be pulled out without spreading the vascular wall at the opening P2. In other words, if the electrode support 25 is pulled out as-is from the opening P2, the electrode support 25 expands at the opening P2, spreading and damaging the vascular wall at the opening P2. However, in the present embodiment, the electrode support 25 is pulled out in the state of being stored in the removal sheath 60 having a predetermined external diameter, and can be removed without placing a burden on the patient P. Once the electrode support 25 is removed together with the removal sheath 60, the operator carries out generally practiced hemostasis, such as suturation and astriction, on the skin of the tapping position. The treatment is thus completed.

The nerve stimulation apparatus of the present embodiment produces the following effects.

(1) The nerve stimulation apparatus 90 of the present embodiment includes: the stimulation electrode portions 10, 11 that transfer electric energy from inside of a blood vessel to a vagus nerve outside of the blood vessel; the heartbeat detection electrodes 15, 16 that detect heartbeat information inside the blood vessel; the stimulus output generating circuit 72 that is connected to the stimulation electrode portions 10, 11 through a conductor and outputs the electric energy to the stimulation electrode portions 10, 11; the heart rate detection circuit 73 that is connected to the heartbeat detection electrodes 15, 16 through a conductor and detects a heart rate on the basis of the heartbeat information detected by the heartbeat detection electrodes 15, 16; the heartbeat status determination circuit 74 that determines a heartbeat status on the basis of the heart rate detected by the heart rate detection. circuit 73; and the notification member 75 that notifies the heartbeat status determined by the heartbeat status determination circuit 74, in which the heartbeat status determination circuit 74 calculates a heart rate reduction rate on the basis of a pre-stimulation heart rate HR_base detected by the heart rate detection circuit 73 before the stimulation output generating circuit 72 starts outputting the electric energy, and an in-stimulation heart rate HR_st detected by the heart rate detection circuit 73 while the stimulation output generating circuit 72 outputs the electric energy, makes the notification member notify the heartbeat status in a case in which at least the heart rate reduction rate is less than a first threshold value (for example, 20%), and stops the stimulus output generating circuit 72 from outputting the electric energy in a case in which the heart rate reduction rate is no less than the first threshold value (for example, 20%). As a result, a nerve stimulation apparatus of greater safety can be provided.

(2) The heartbeat status determination circuit 74 of the present embodiment calculates the heart rate reduction rate by the following equation W.

heart rate reduction rate (%)=((HR_base)−(HR_st))/(HR_base)) ×100 . . . (1)

The notification of the heartbeat status by the notification member 75 and stop of electric energy output can thus be carried out more appropriately. Safety can thus be improved.

(3) In the present embodiment, the first threshold value is no less than 15% and no greater than 25%. The notification of the heartbeat status by the notification member 75 and stop of electric energy output can thus be carried out more appropriately. Safety can thus be improved.

(4) In a case in which the heart rate reduction rate is no less than a second threshold value smaller than the first threshold value and less than the first threshold value, the heartbeat status determination circuit 74 of the present embodiment makes the notification member 75 notify that the heart rate reduction rate is in the warning state. As a result, a notification of the warning state of the heart rate reduction rate can be made more appropriately. Safety can thus be improved.

(5) The notification member 75 of the present embodiment notifies, in accordance with the heart rate reduction rate, in an ascending order of the heart rate reduction rate: a first notification state indicating that an intensity of stimulation or the vagus nerve is insufficient; a second notification state indicating that the intensity of stimulation of the vagus nerve is appropriate; and a third notification state indicating that the heart rate reduction rate is in the warning state. As a result, the plurality of notification states can be separately notified in accordance with the heart rate reduction rate. The operator and the like can thus assess the situation more easily.

(6) The notification member 75 of the present embodiment is provided with a plurality of LEDs of different colors; and the heartbeat status determination circuit 74 turns on one of the plurality of LEDs of a color corresponding to the heart rate reduction rate. As a result, the plurality of notification states can be separately notified in accordance with the heart rate reduction rate. The operator and the like can thus assess the situation more easily.

(7) The heart rate detection circuit 73 of the present embodiment detects the in-stimulation heart rate HR_st on the basis of the heartbeat information detected after a lapse of a determination stand-by period since the stimulation output generating circuit 72 starts outputting the electric energy, until the stimulation output generating circuit 72 stops outputting the electric energy. The heart rate starts to decrease immediately after starting applying the stimulus, and takes time to reach the minimum heart rate. Therefore, the heart rate during the decreasing period immediately after starting applying the stimulus is not counted in calculation of the in-stimulation heart rate HR_st. The in-stimulation heart rate can thus be detected more accurately.

(8) In the present embodiment, the determination stand-by period is no less than 3 sec. Since the time required to reach the minimum heart rate is about 3 sec from the start of application of the stimulus, the determination stand-by period is preferably no less than 3 sec.

(9) The stimulus output generating circuit 72 has a plurality of output modes for outputting a stimulating pulse as the electric energy, the plurality of output modes including: a continuous stimulation mode in which the stimulating pulse is continuously output; and an intermittent stimulation mode in which a stimulating pulse group is intermittently output. It is desirable that the intravascular stimulation electrode is opposed to the vagus nerve, in order to efficiently transmit the electric energy to the vagus nerve outside of the blood vessel. The opposite positional relationship is determined through a decrease in heart rate. The operator can more easily detect the decrease in heart rate in the continuous stimulation mode. On the other hand, in light of a curative effect, the intermittent stimulation mode can be employed. The operation of intermittently decreasing the heart rate places less burden on the patient.

(10) The method of the present embodiment stimulates a vagus nerve outside of a blood vessel from inside of the blood vessel by means of the nerve stimulation apparatus 90 comprising: the stimulation electrode portions 10, 11 that transfer electric energy from inside of a blood vessel to a vagus nerve outside of the blood vessel; the heartbeat detection electrodes 15, 16 that detect heartbeat information inside the blood vessel; the notification member 75, the method comprising: calculating a heart rate reduction rate on the basis of a pre-stimulation heart rate HR base based on the heartbeat information detected by the heartbeat detection electrodes 15, 16 prior to output of the electric energy to the stimulation electrode portions 10, 11 and an in-stimulation heart rate HR_st based on the heartbeat information detected by the heartbeat detection electrodes 15, 16 during output of the electric energy to the stimulation electrode portions 10, 11; making the notification member 75 notify the heartbeat status in a case in which at least the heart rate reduction rate is less than the first threshold value; and stopping output of the electric energy to the stimulation electrode portions 10, 11 in a case in which the heart rate reduction. rate is no less than the first threshold value. As a result, nerve stimulation of greater safety can be provided.

(11) In the method of the present embodiment, the nerve stimulation apparatus 90 outputs the stimulating pulse in the continuous stimulation mode during positioning of the stimulation electrode portions 10, 11, and outputs the stimulating pulse in the intermittent stimulation mode during a treatment period after the positioning of the stimulation electrode portions 10, 11. It is desirable that the intravascular stimulation electrode is opposed to the vagus nerve, in order to efficiently transmit the electric energy to the vagus nerve outside of the blood vessel. The opposite positional relationship is determined through a decrease in heart rate. The operator can more easily detect the decrease in heart rate in the continuous stimulation mode. On the other hand, in light of a curative effect, the intermittent stimulation mode can be employed. The operation of intermittently decreasing the heart rate places less burden on the patient.

The preferred embodiment of the present invention. has been described above; however, the present invention is not in any way limited to the embodiments. Addition, omission substitution and other changes of configuration(s) can be made within the range not to depart from the spirit of the present invention. In addition, the present invention is not limited to the foregoing descriptions and is limited only by the Claims.

### EXPLANATION OF REFERENCE NUMERALS

1 Electrical stimulation electrode (medical instrument)
10, 11 Stimulation electrode portion (intravascular stimulation electrode)
15, 16 Heartbeat detection electrode (intravascular heartbeat detection electrode)
20 mead portion (linear body)
25 Electrode support (biasing member)
50 Operation sheath
60 Removal sheath
70 Electrical stimulation device
71 Operation unit
72 Stimulus output generating circuit
73 Heart rate detection circuit
74 Heartbeat status determination circuit
75 Notification member
90 Nerve stimulation apparatus
100 Medical instrument set
P3 Vena jugularis interna (blood vessel)
P5 Superior vena cava (blood vessel)
P6 Vagus nerve

What is claimed:

1. A nerve stimulation apparatus comprising:
an intravascular stimulation electrode that transfers electric energy from inside of a blood vessel to a vagus nerve outside of the blood vessel;
an intravascular heartbeat detection electrode that detects heartbeat information inside the blood vessel;
a stimulus output generating circuit that is connected to the intravascular stimulation electrode through a conductor and outputs the electric energy to the intravascular stimulation electrode;
a heart rate detection circuit that connected to the intravascular heartbeat detection electrode through a conductor and detects a heart rate on the basis of the heartbeat information detected by the intravascular heartbeat detection electrode;
a heartbeat status determination circuit that determines a heartbeat status on the basis of the heart rate detected by the heart rate detection circuit; and
a notification member that notifies the heartbeat status determined by the heartbeat status determination circuit,
wherein
the heartbeat status determination circuit:
calculates a heart rate reduction rate on the basis of a pre-stimulation heart rate HR_base detected by the heart rate detection circuit before the stimulus output generating circuit starts outputting the electric energy, and an in-stimulation heart rate HR_st detected by the heart rate detection circuit while the stimulus output generating circuit outputs the electric energy;
makes the notification member notify the heartbeat status in a case in which at least the heart rate reduction rate is less than a first threshold value; and
stops the stimulus output generating circuit from outputting the electric energy in a case in which the heart rate reduction rate is no less than the first threshold value.

2. The nerve stimulation apparatus according to claim 1, wherein the heartbeat status determination circuit calculates the heart rate reduction rate by the following equation (1).
heart rate reduction rate (%)=((HR_base)−(HR_st))/(HR_base)) ×100 . . . (1)

3. The nerve stimulation apparatus according to claim wherein the first threshold value is no less than 15% and no greater than 25%.

4. The nerve stimulation apparatus according to claim 1, wherein, in a case in which the heart rate reduction rate is no less than a second threshold value smaller than the first threshold value and less than the first threshold value, the heartbeat status determination circuit makes the notification member notify that the heart rate reduction rate in a warning state.

5. The nerve stimulation apparatus according to claim 1, wherein the notification member notifies, in accordance with the heart rate reduction rate, in an ascending order of the heart rate reduction rate: a first notification state indicating that an intensity of stimulation of the vagus nerve is insufficient; a second notification state indicating that the intensity of stimulation of the vagus nerve is appropriate; and a third notification state indicating that the heart rate reduction rate is in the warning state.

6. The nerve stimulation apparatus according to claim 1, wherein:
the notification member is provided with a plurality of LEDs of different colors; and
the heartbeat status determination circuit turns on one of the plurality of LEDs of a color corresponding to the heart rate reduction rate.

7. The nerve stimulation apparatus according to claim 1, wherein the heart rate detection circuit detects the in-stimulation heart rate HR_st on the basis of the heartbeat information detected after a lapse of a determination stand-by period since the stimulus output generating circuit starts outputting the electric energy, until the stimulus output generating circuit stops outputting the electric energy.

8. The nerve stimulation apparatus according to claim 7, wherein the determination stand-by period is no less than 3 seconds.

9. The nerve stimulation apparatus according to claim 1, wherein the stimulus output generating circuit has a plurality of output modes for outputting a stimulating pulse, the plurality of output modes comprising: a continuous stimulation mode in which the stimulating pulse is continuously output; and an intermittent stimulation mode in which a stimulating pulse group is intermittently output.

10. A method of stimulating a vagus nerve outside of a blood vessel from inside of the blood vessel by means of a nerve stimulation apparatus comprising:
an intravascular stimulation electrode that transfers electric energy from inside of a blood vessel to a vagus nerve outside of the blood vessel; an intravascular heartbeat detection electrode that detects heartbeat information inside the blood vessel; and a notification member, the method comprising:
calculating a heart rate reduction rate on the basis of a pre-stimulation heart rate HR_base based on the heartbeat information detected by the intravascular heartbeat detection electrode prior to output of the electric energy to the intravascular stimulation electrode and an in-stimulation heart rate HR_st based on the heartbeat information detected by the intravascular heartbeat detection electrode during output of the electric energy to the intravascular stimulation electrode;
making the notification member notify the heartbeat status in a case in which at least the heart rate reduction rate is less than a first threshold value; and
stopping output of the electric energy to the intravascular stimulation electrode in a case in which the heart rate reduction rate is no less than the first threshold value.

11. The method according to claim 10, wherein the nerve stimulation apparatus has a plurality of output modes for outputting a stimulating pulse, the plurality of output modes comprising: a continuous stimulation mode in which the stimulating pulse is continuously output; and an intermittent stimulation mode in which a stimulating pulse group is intermittently output,
and the nerve stimulation apparatus outputs the stimulating pulse in the continuous stimulation mode during positioning of the intravascular stimulation electrode, and outputs the stimulating pulse in the intermittent stimulation mode during a treatment period after the positioning of the intravascular stimulation electrode.
